(19) United States
(12) Patent Application Publication
(10) Pub. No.: US 2013/0074201 A1
(43) Pub. Date: Mar. 21, 2013

(54) CANCER-SPECIFIC GENETIC REARRANGEMENTS

(71) Applicants: Scott Kern, Hunt Valley, MD (US); Francesca Scrimieri, Washington, DC (US)

(72) Inventors: Scott Kern, Hunt Valley, MD (US); Francesca Scrimieri, Washington, DC (US)

(73) Assignee: THE JOHNS HOPKINS UNIVERSITY, Baltimore, MD (US)

(21) Appl. No.: 13/621,460

(22) Filed: Sep. 17, 2012

Related U.S. Application Data
(60) Provisional application No. 61/535,543, filed on Sep. 16, 2011.

(51) Int. Cl. A61K 39/395; C12N 15/63; C12N 1/21; C12N 1/19; C12N 5/10; C12P 21/02; G01N 33/574; C12Q 1/68; C12N 15/113; C07K 16/18; A61K 31/7088; A61K 48/00; A61P 35/00; A01K 67/027; C12N 15/12

(52) U.S. Cl. 800/18; 536/23.5; 435/320.1; 435/252.33; 435/254.21; 435/348; 435/325; 435/357; 435/367; 435/365; 435/69.6; 435/6.12; 435/7.1; 435/6.13; 536/24.5; 530/387.9; 424/172.1; 514/44 A

(57) ABSTRACT

The present invention relates to the field of cancer. More specifically, the present invention provides compositions and methods useful for treating cancer characterized by the expression of mutant FAM190A proteins. In a specific embodiment, a method for treating a patient having a cancer characterized by a FAM190A intragenic rearrangement comprises the step of administering to the patient an agent that inhibits a biological function or reduces the level or expression of the FAM190A protein.

### CROSS-REFERENCE TO RELATED APPLICATIONS

This application claims the benefit of U.S. Provisional Application No. 61/535,543, filed Sep. 16, 2011; which is incorporated herein by reference in its entirety.

### STATEMENT OF GOVERNMENTAL INTEREST

This invention was made with U.S. government support under grant no. CA62924 and grant no. CA134292. The U.S. government has certain rights in the invention.

### FIELD OF THE INVENTION

The present invention relates to the field of cancer. More specifically, the present invention provides compositions and methods useful for treating cancer characterized by the expression of mutant FAM190A proteins.

### INCORPORATION-BY-REFERENCE OF MATERIAL SUBMITTED ELECTRONICALLY

This application contains a sequence listing. It has been submitted electronically via EFS-Web as an ASCII text file entitled “P11005-06_ST25.txt.” The sequence listing is 318,577 bytes in size, and was created on Sep. 13, 2012. It is hereby incorporated by reference in its entirety.

### BACKGROUND OF THE INVENTION

Genomic alterations play an important role in the pathogenesis of many diseases because they have the potential of creating an abnormal fusion of two genes or to activate proto-oncogenes. This is especially true in hematologic malignancies and some sarcomas, where the initiation of the malignant process often involves chromosomal rearrangements that activate various oncogenes. Many of the chromosomal rearrangements in leukemia and lymphoma are thought to result from errors in the physiologic process of immunoglobulin or T-cell receptor gene rearrangement during normal B-cell and T-cell development. In astrocytic gliomas, the allelic deletions in the intragenic region of EGFR (called EGFR vIII) occurred in association with gene amplification. This indicates the probable involvement of EGFR vIII as an important event in the development of gene amplification. EGFRvIII is also an attractive target antigen for cancer immunotherapy because it is not expressed in normal tissue and because cells producing EGFRvIII have an enhanced oncogenic properties (1).

### SUMMARY OF THE INVENTION

The present invention is based, at least in part, on the discovery of a novel and recurrent oncogene existing in multiple tumor types. Oncogenes are cancer-specific gene mutations, recurring in multiple patients, that result in functioning, but abnormal, RNA transcripts and proteins. Discovering such mutations is clinically useful for the mutant genes, transcripts and proteins can be identified in clinical samples to aid in making a diagnosis of a neoplasm, to indicate a particular diagnostic classification, to permit improved prognostication, and to provide an assayable marker for monitoring cancer burden during and after therapy.

Knowledge of an oncogene can be used to create reagents for scientific and pharmacologic research such as transgenic mice and oncogene-specific antibodies, and can be targeted by oncogene-specific drugs to treat the patient. Most rearrangements and other genetic mutations of neoplasia do not create oncogenes, for they are known to disrupt gene function by interrupting normal RNA transcription and full protein translation, and most are non-recurrent among different patients. A variety of individually distinct genetic mutations create rearrangements that, when the associated RNA transcript is spliced to form the mature mRNA species, produce the identical and novel open reading frame not found in normal tissues.

The present invention provides methods of detecting or measuring the quantity of the intragenically rearranged genome, mutant transcript or mutant protein in a patient-derived sample for detecting whether or not neoplasm exists or for monitoring a neoplasm's severity, as well as in a tumor sample for diagnostic classification. These methods may include binding complementary oligonucleotides on the mutant sequences or on flanking sequences in order to copy the site of mutation, or binding the mutant peptide to antibodies, or determining the nucleotide (for DNA or cDNA) or amino acid sequence at the site of the mutation.

The present invention also provides methods for detecting the rearranged genome, mutant transcript and mutant protein in cell lines and animals for research. The present invention also provides:

Enriched, isolated, purified forms of the mutant protein or mutant peptide fragments, including dry, crystallized or in solution, whether or not including minor constituents of impurities or additives.

A purified polyclonal or monoclonal antibody specific to the mutant peptide sequence.

A polynucleotide complementary to the mutant nucleotide sequence.

A transgenic non-human animal (including, but not limited to, mouse, rat and fish) in which the human mutant sequence or substantially identical or functionally equivalent sequence is introduced with an appropriate promoter or at the site of a native promoter and producing the animal protein.

A non-human animal (including, but not limited to, mouse, rat and fish) in which two adjacent internal exons of the murine gene are genetically inactivated to create a specific mutant transcript and protein.

A cell line engineered to acquire the mutant gene, or express a peptide or protein containing the mutant amino acid sequence or engineered by gene knock-out technology to lack the naturally-occurring mutant gene.

Methods of screening a panel or library of test compounds and employing animals, cells or cell-free reagents in solution, containing the mutant peptide sequence, where the interaction of the compound and the mutant peptide or protein is detected, or where the mutant peptide is made to contact a second protein or peptide (such as an aptamer) to form a complex and a compound's ability to disrupt the complex is detected, or where changes in the NMR spectrum or molecular motion of the mutant or wildtype gene is detected.

Methods of treating a neoplasm in a patient or vertebrate animal by inhibiting the expression or function of the mutant protein (as by administering a drug or antibody to contact the mutant protein or mutant protein fragment, or a nucleotide sequence complementary (hybridizing) to the mutant transcript, or a double-stranded RNA (using siRNA technology) containing the mutant sequence and it complementary sequence).

Methods of treating a neoplastic condition by inhibiting the expression or function of the wild-type protein.

Methods of treating a neoplastic condition by vaccinating the patient against the mutant amino acid sequence (inoculating a patient with a preparation containing the mutant peptide or cells engineered to express the mutant peptide or a fusion of the mutant peptide and an immunogenic peptide).

Plasmids or other expression vectors containing the mutant nucleotide sequence. These vectors may express a strand of the mutant transcript, a sequence complementary to that sequence, or a double-stranded RNA transcript containing sequences of both strands.

Kits for the detection of mutant nucleotide or amino acid sequences.

Nucleotide sequences for expression and containing an intron within the mutant sequence are interchangeable with those containing a contiguous sequence of the mutant nucleotide sequence. Nucleotide sequences for detection and containing a detectable moiety are interchangeable with those lacking such a moiety. Small peptides containing the mutant amino acid sequence are interchangeable with large peptides, full-length protein, and peptides and proteins fused to detectable peptide tags.

In one embodiment, an isolated nucleic acid molecule comprises a sequence as shown in SEQ ID NO:2. In another embodiment, an isolated nucleic acid molecule comprises a sequence as shown in SEQ ID NO:3. In an alternative embodiment, an isolated nucleic acid molecule comprises a sequence as shown in SEQ ID NO:4. In a specific embodiment, an isolated nucleic acid molecule comprises a sequence as shown in SEQ ID NO:5. In a further embodiment, an isolated nucleic acid molecule comprises a sequence as shown in SEQ ID NO:6. In yet another embodiment, an isolated nucleic acid molecule comprises a sequence as shown in SEQ ID NO:7. In a specific embodiment, an isolated nucleic acid molecule comprises a sequence as shown in SEQ ID NO:8. The present invention also provides an isolated nucleic acid molecule comprising a sequence as shown in SEQ ID NO:9. In one embodiment, an isolated nucleic acid molecule comprises a sequence as shown in SEQ ID NO:10. In a particular embodiment, an isolated nucleic acid molecule comprises a sequence as shown in SEQ ID NO:11. In another embodiment, an isolated nucleic acid molecule comprises a sequence as shown in SEQ ID NO:12. In a further embodiment, an isolated nucleic acid molecule comprises a sequence as shown in SEQ ID NO:13. In yet another embodiment, an isolated nucleic acid molecule comprises a sequence as shown in SEQ ID NO:14. In a certain embodiment, an isolated nucleic acid molecule comprises a sequence as shown in SEQ ID NO:15. In a specific embodiment, an isolated nucleic acid molecule comprises a sequence as shown in SEQ ID NO:16.

In another embodiment, an isolated amino acid molecule comprises a sequence as shown in SEQ ID NO:18. In a further embodiment, an isolated amino acid molecule comprises a sequence as shown in SEQ ID NO:19. In yet another embodiment, an isolated amino acid molecule comprises a sequence as shown in SEQ ID NO:20. In a specific embodiment, an isolated amino acid molecule comprises a sequence as shown in SEQ ID NO:21. In an alternative embodiment, an isolated amino acid molecule comprises a sequence as shown in SEQ ID NO:22. In one embodiment, an isolated amino acid molecule comprises a sequence as shown in SEQ ID NO:23. In another embodiment, an isolated amino acid molecule comprises a sequence as shown in SEQ ID NO:24. In a further embodiment, an isolated amino acid molecule comprises a sequence as shown in SEQ ID NO:25. In yet another embodiment, an isolated amino acid molecule comprises a sequence as shown in SEQ ID NO:26. In an alternative embodiment, an isolated amino acid molecule comprises a sequence as shown in SEQ ID NO:27.

The present invention further provides isolated nucleic acid molecule that encode the amino acid sequences of mutant FAM190A proteins. In one embodiment, an isolated nucleic acid molecule encodes the amino acid sequence as shown in SEQ ID NO:18. In another embodiment, an isolated nucleic acid molecule encodes the amino acid sequence as shown in SEQ ID NO:19. In yet another embodiment, an isolated nucleic acid molecule encodes the amino acid sequence as shown in SEQ ID NO:20. In a further embodiment, an isolated nucleic acid molecule encodes the amino acid sequence as shown in SEQ ID NO:21. In an alternative embodiment, an isolated nucleic acid molecule encodes the amino acid sequence as shown in SEQ ID NO:22. In a particular embodiment, an isolated nucleic acid molecule encodes the amino acid sequence as shown in SEQ ID NO:23. In a specific embodiment, an isolated nucleic acid molecule encodes the amino acid sequence as shown in SEQ ID NO:24. In another embodiment, an isolated nucleic acid molecule encodes the amino acid sequence as shown in SEQ ID NO:25. In yet another embodiment, an isolated nucleic acid molecule encodes the amino acid sequence as shown in SEQ ID NO:26. In a further embodiment, an isolated nucleic acid molecule encodes the amino acid sequence as shown in SEQ ID NO:27.

The present invention also provides murine nucleic acid sequences encoding mutant FAM190A protein. In one embodiment, an isolated nucleic acid molecule comprises a sequence as shown in SEQ ID NO:29. In another embodiment, an isolated nucleic acid molecule comprises a sequence as shown in SEQ ID NO:30. In yet another embodiment, an isolated nucleic acid molecule comprises a sequence as shown in SEQ ID NO:31. In a further embodiment, an isolated nucleic acid molecule comprises a sequence as shown in SEQ ID NO:32. In an alternative embodiment, an isolated nucleic acid molecule comprises a sequence as shown in SEQ ID NO:33. In a specific embodiment, an isolated nucleic acid molecule comprises a sequence as shown in SEQ ID NO:34. In another embodiment, an isolated nucleic acid molecule comprises a sequence as shown in SEQ ID NO:35. In yet another embodiment, an isolated nucleic acid molecule comprises a sequence as shown in SEQ ID NO:36. In a further embodiment, an isolated nucleic acid molecule comprises a sequence as shown in SEQ ID NO:37. In a specific embodiment, an isolated nucleic acid molecule comprises a sequence as shown in SEQ ID NO:38.

The present invention also provides murine amino acid sequences for mutant FAM190A protein. In one embodiment, an isolated amino acid molecule comprises a sequence as shown in SEQ ID NO:39. In another embodiment, an isolated amino acid molecule comprises a sequence as shown in SEQ ID NO:40. In yet another embodiment, an isolated amino acid molecule comprises a sequence as shown in SEQ ID NO:41. In a further embodiment, an isolated amino acid molecule comprises a sequence as shown in SEQ ID NO:42. In an alternative embodiment, an isolated amino acid molecule comprises a sequence as shown in SEQ ID NO:43. In a specific embodiment, an isolated amino acid molecule comprises a sequence as shown in SEQ ID NO:44. In another embodiment, an isolated amino acid molecule comprises a sequence as shown in SEQ ID NO:45. In yet another embodiment, an isolated amino acid molecule comprises a sequence as shown in SEQ ID NO:46. In a further embodiment, an isolated amino acid molecule comprises a sequence as shown in SEQ ID NO:47. In a specific embodiment, an isolated amino acid molecule comprises a sequence as shown in SEQ ID NO:48.

The present invention further provides isolated nucleic acid molecule that encode the amino acid sequences of murine mutant FAM190A proteins. In one embodiment, an isolated nucleic acid molecule encodes the amino acid sequence as shown in SEQ ID NO:39. In another embodiment, an isolated nucleic acid molecule encodes the amino acid sequence as shown in SEQ ID NO:40. In yet another embodiment, an isolated nucleic acid molecule encodes the amino acid sequence as shown in SEQ ID NO:41. In a further embodiment, an isolated nucleic acid molecule encodes the amino acid sequence as shown in SEQ ID NO:42. In an alternative embodiment, an isolated nucleic acid molecule encodes the amino acid sequence as shown in SEQ ID NO:43. In a specific embodiment, an isolated nucleic acid molecule encodes the amino acid sequence as shown in SEQ ID NO:44. In another embodiment, an isolated nucleic acid molecule encodes the amino acid sequence as shown in SEQ ID NO:45. In yet another embodiment, an isolated nucleic acid molecule encodes the amino acid sequence as shown in SEQ ID NO:46. In a further embodiment, an isolated nucleic acid molecule encodes the amino acid sequence as shown in SEQ ID NO:47. In another embodiment, an isolated nucleic acid molecule encodes the amino acid sequence as shown in SEQ ID NO:48.

The present invention also provides expression vectors. In one embodiment, an expression vector comprises a nucleic acid molecule comprising SEQ ID NO:2. In another embodiment, an expression vector comprises a nucleic acid molecule comprising SEQ ID NO:3. In yet another embodiment, an expression vector comprises a nucleic acid molecule comprising SEQ ID NO:4. In a further embodiment, an expression vector comprises a nucleic acid molecule comprising SEQ ID NO:5. In an alternative embodiment, an expression vector comprises a nucleic acid molecule comprising SEQ ID NO:6. In a specific embodiment, an expression vector comprises a nucleic acid molecule comprising SEQ ID NO:7. In another embodiment, an expression vector comprises a nucleic acid molecule comprising SEQ ID NO:8. In yet another embodiment, an expression vector comprises a nucleic acid molecule comprising SEQ ID NO:9. In a further embodiment, an expression vector comprises a nucleic acid molecule comprising SEQ ID NO:10. In one embodiment, an expression vector comprises a nucleic acid molecule comprising SEQ ID NO:11. In another embodiment, an expression vector comprises a nucleic acid molecule comprising SEQ ID NO:12. In yet another embodiment, an expression vector comprises a nucleic acid molecule comprising SEQ ID NO:13. In a further embodiment, an expression vector comprises a nucleic acid molecule comprising SEQ ID NO:14. In one embodiment, an expression vector comprises a nucleic acid molecule comprising SEQ ID NO:15. In another embodiment, an expression vector comprises a nucleic acid molecule comprising SEQ ID NO:16. The present invention also provides host cells transformed with an expression vector comprising a nucleic acid molecule selected from the group consisting of SEQ ID NOS:2-16.

In one embodiment, an expression vector comprises a nucleic acid molecule comprising SEQ ID NO:29. In another embodiment, an expression vector comprises a nucleic acid molecule comprising SEQ ID NO:30. In yet another embodiment, an expression vector comprises a nucleic acid molecule comprising SEQ ID NO:31. In a further embodiment, an expression vector comprises a nucleic acid molecule comprising SEQ ID NO:32. In an alternative embodiment, an expression vector comprises a nucleic acid molecule comprising SEQ ID NO:33. In a specific embodiment, an expression vector comprises a nucleic acid molecule comprising SEQ ID NO:34. In another embodiment, an expression vector comprises a nucleic acid molecule comprising SEQ ID NO:35. In yet another embodiment, an expression vector comprises a nucleic acid molecule comprising SEQ ID NO:36. In a further embodiment, an expression vector comprises a nucleic acid molecule comprising SEQ ID NO:37. In one embodiment, an expression vector comprises a nucleic acid molecule comprising SEQ ID NO:38. The present invention also provides host cells transformed with an expression vector comprising a nucleic acid molecule selected from the group consisting of SEQ ID NOS:29-38.

The present invention also provides a host cell comprising a cDNA which encodes an immunogenic agent, wherein the host cell expresses the immunogenic agent. In one embodiment, the immunogenic agent comprises the amino acid shown in SEQ ID NO:18. In another embodiment, the immunogenic agent comprises the amino acid shown in SEQ ID NO:19. In yet another embodiment, the immunogenic agent comprises the amino acid shown in SEQ ID NO:20. In a further embodiment, the immunogenic agent comprises the amino acid shown in SEQ ID NO:21. In an alternative embodiment, the immunogenic agent comprises the amino acid shown in SEQ ID NO:22. In a specific embodiment, the immunogenic agent comprises the amino acid shown in SEQ ID NO:23. In another embodiment, the immunogenic agent comprises the amino acid shown in SEQ ID NO:24. In yet another embodiment, the immunogenic agent comprises the amino acid shown in SEQ ID NO:25. In a further embodiment, the immunogenic agent comprises the amino acid shown in SEQ ID NO:26. In another embodiment, the immunogenic agent comprises the amino acid shown in SEQ ID NO:27.

The present invention also provides a host cell comprising a cDNA which encodes an immunogenic agent, wherein the host cell expresses the immunogenic agent. In one embodiment, the immunogenic agent comprises the amino acid shown in SEQ ID NO:39.

In another embodiment, the immunogenic agent comprises the amino acid shown in SEQ ID NO:40. In yet another embodiment, the immunogenic agent comprises the amino acid shown in SEQ ID NO:41. In a further embodiment, the immunogenic agent comprises the amino acid shown in SEQ ID NO:42. In an alternative embodiment, the immunogenic agent comprises the amino acid shown in SEQ ID NO:43. In a specific embodiment, the immunogenic agent comprises the amino acid shown in SEQ ID NO:44. In another embodiment, the immunogenic agent comprises the amino acid shown in SEQ ID NO:45. In yet another embodiment, the immunogenic agent comprises the amino acid shown in SEQ ID NO:46. In a further embodiment, the immunogenic agent comprises the amino acid shown in SEQ ID NO:47. In another embodiment, the immunogenic agent comprises the amino acid shown in SEQ ID NO:48.

The present invention also provides methods for generating antibodies. In one embodiment, a method of generating antibodies comprises the steps of (a) culturing a host cell comprising a cDNA encoding an immunogenic agent whereby the immunogenic agent is expressed in the host cell, and (b) administering the immunogenic agent to a non-human animal, wherein the animal produces antibodies which specifically bind to the immunogenic agent. In one embodiment, the immunogenic agent comprises the amino acid shown in SEQ ID NO:18. In another embodiment, the immunogenic agent comprises the amino acid shown in SEQ ID NO:19. In yet another embodiment, the immunogenic agent comprises the amino acid shown in SEQ ID NO:20. In a further embodiment, the immunogenic agent comprises the amino acid shown in SEQ ID NO:21. In an alternative embodiment, the immunogenic agent comprises the amino acid shown in SEQ ID NO:22. In a specific embodiment, the immunogenic agent comprises the amino acid shown in SEQ ID NO:23. In another embodiment, the immunogenic agent comprises the amino acid shown in SEQ ID NO:24. In yet another embodiment, the immunogenic agent comprises the amino acid shown in SEQ ID NO:25. In a further embodiment, the immunogenic agent comprises the amino acid shown in SEQ ID NO:26. In another embodiment, the immunogenic agent comprises the amino acid shown in SEQ ID NO:27.

In a specific embodiment, the immunogenic agent is administered in a purified protein preparation. In other embodiments, the method further comprises (c) collecting B cells from the animal; (d) fusing the B cells with myeloma cells to make hybridomas; and (e) collecting the antibodies from the hybridomas. In certain embodiments, the non-human animal is a mouse. The present invention also provides antibody compositions made from the process described herein.

The present invention also provides methods for diagnosing a disease characterized by, or otherwise implicating, a mutant FMA protein. In certain embodiments, the disease is cancer. In a specific embodiment, a method for diagnosing cancer or a likelihood thereof in a patient comprises the steps of (a) obtaining a biological sample from the patient; (b) subjecting the sample to an assay for detecting any of the FAM190A mutant proteins selected from the group consisting of SEQ ID NOS: 18-25; and (d) determining that the patient has cancer or a likelihood thereof if a FAM190A mutant protein is detected. In particular embodiments, the method comprises detecting one or more FAM190A mutant proteins selected from the group consisting of SEQ ID NOS:18-25.

In alternative embodiments, a method for diagnosing cancer or a likelihood thereof in a patient comprises the steps of (a) obtaining a biological sample from the patient; (b) subjecting the sample to an assay for detecting any of the FAM190A mutant nucleic acid sequences selected from the group consisting of SEQ ID NOS: 2-10; and (d) determining that the patient has cancer or a likelihood thereof if a FAM190A mutant nucleic acid sequence is detected.

The present invention also provides methods for identifying an agent useful for treating cancer in a patient. In one embodiment, the method comprises the steps of (a) providing a cell which expresses FAM190A protein selected from the group consisting of SEQ ID NOS:18-27; (b) exposing the cell to candidate agents; and (c) identifying an agent that inhibits a biological function or reduces the level or expression of the FAM190A protein.

In other embodiments of the present invention, a method for treating a patient having a cancer characterized by a FAM190A intragenic rearrangement comprises the step of administering to the patient an agent that inhibits a biological function or reduces the level or expression of the FAM190A protein. In a specific embodiment, the agent is selected from the group consisting of an antibody, an inhibitory nucleic acid molecule, and a small molecule.

The present invention further provides a transgenic mouse that expresses a mutant FAM190A having an amino acid sequence selected from the group consisting of SEQ ID NOS: 18-27.

In other embodiments, the present invention provides isolated antibodies, or biologically active fragments thereof, which specifically bind a FAM190A mutant protein having an amino acid sequence selected from the group consisting of SEQ ID NOS:18-27. In specific embodiments, the antibody is a monoclonal antibody.

In further embodiments, the present invention provides inhibitory nucleic acids. In a specific embodiment, an inhibitory nucleic acid molecule that is complementary to at least a fragment of a FAM190A nucleic acid molecule selected from the group consisting of SEQ ID NOS:2-16, and that decreases FAM190A expression in a cell. In a more specific embodiment, the nucleic acid molecule is an siRNA. In an alternative embodiment, the nucleic acid molecule is an shRNA.

### BRIEF DESCRIPTION OF THE FIGURES

FIG. 1: Diagram of an intragenic deletion found in the human FAM190A gene (KIAA1680). Each box represents an exon while the line between the boxes represents an intron. The broken line shows the region lost in the cancer rearrangement. The cancer mRNA connects the splice donor site of exon 6 with the splice acceptor site of exon 9. The RNA-to-protein translation joins the CAA (Gln) GAG (Glu) sequence of exon 6 to GGA (Gly) AAA (Lys) sequence of exon 9.

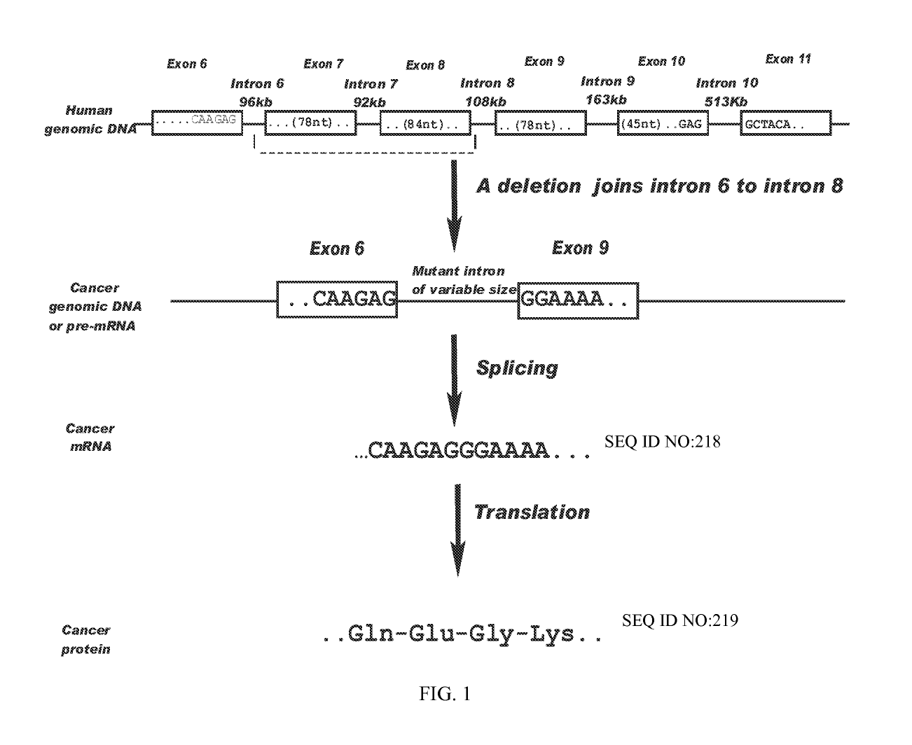

FIG. 2: The cancer mRNA connects the splice donor site of exon 6 with the splice acceptor site of exon 10. The RNA-to-protein translation joins the CAA (Gln) GAG (Glu) sequence of exon 6 to GGT (Gly) TTA (Leu) sequence of exon 10.

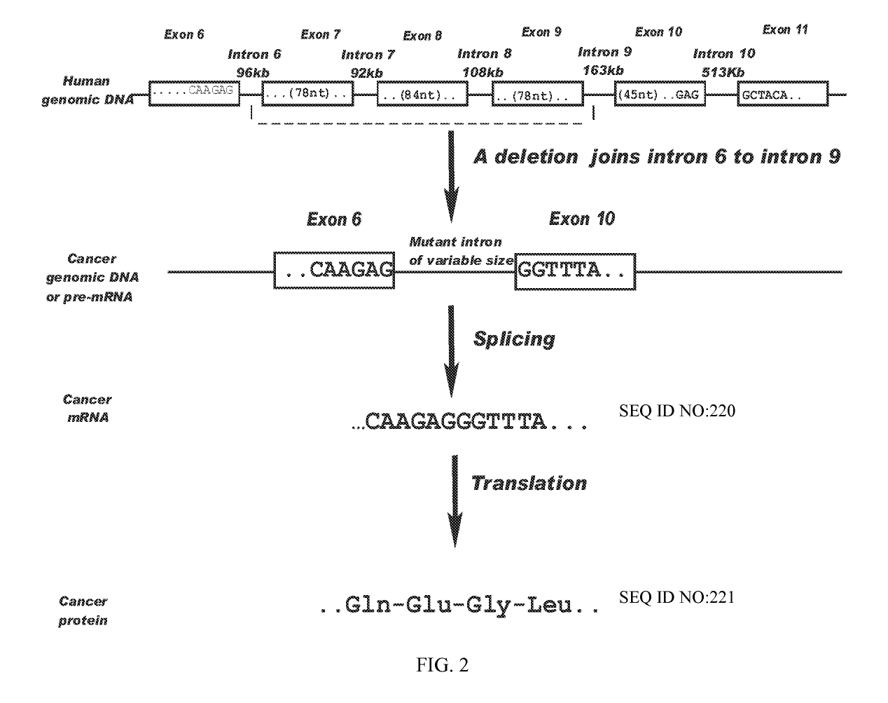

FIG. 3: The cancer mRNA connects the splice donor site of exon 6 with the splice acceptor site of exon 11. The RNA-to-protein translation joins the CAA (Gln) GAG (Glu) sequence of exon 6 to GCT (Ala) ACA (Thr) sequence of exon 11.

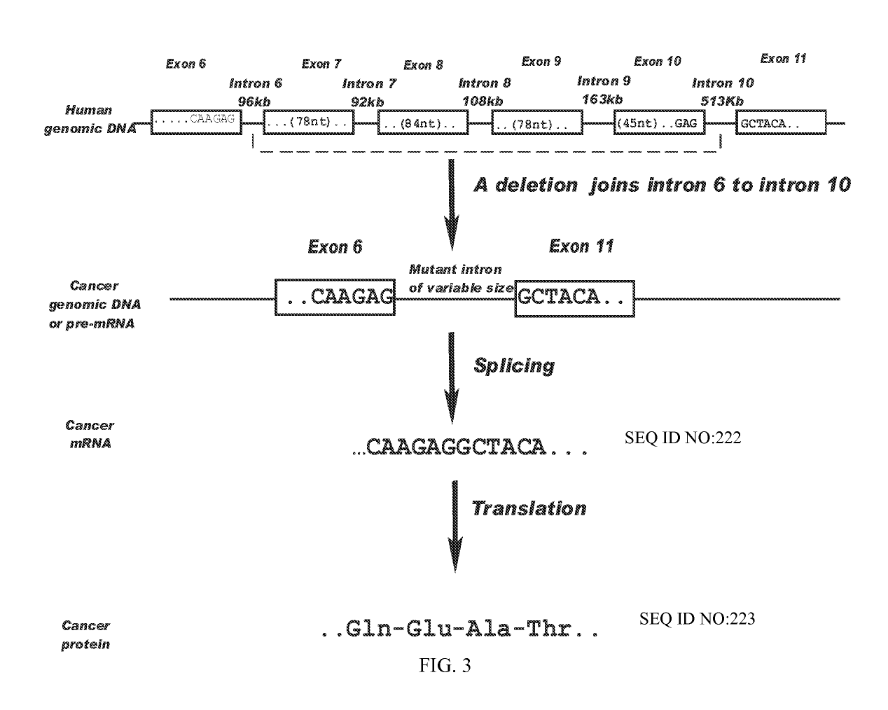

FIG. 4: The cancer mRNA connects the splice donor site of exon 7 with the splice acceptor site of exon 10. The RNA-to-protein translation joins the TTC (Phe) AAG (Lys) sequence of exon 7 to GGT (Gly) TTA (Leu) sequence of exon 10.

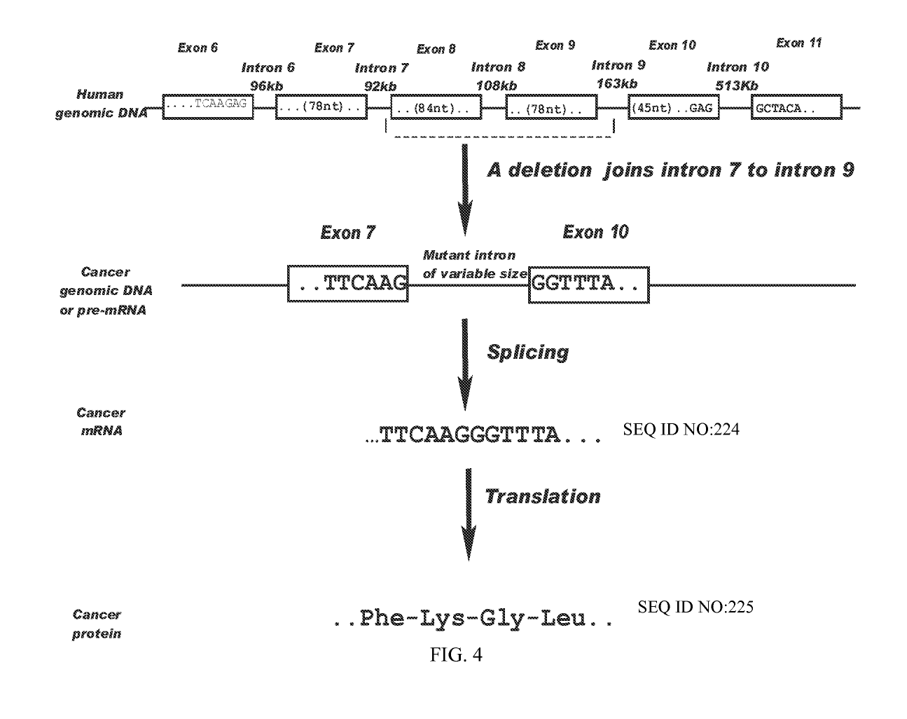

FIG. 5: The cancer mRNA connects the splice donor site of exon 7 with the splice acceptor site of exon 11. The RNA-to-protein translation joins the TTC (Phe) AAG (Lys) sequence of exon 7 to GCT (Ala) ACA (Thr) sequence of exon 11.

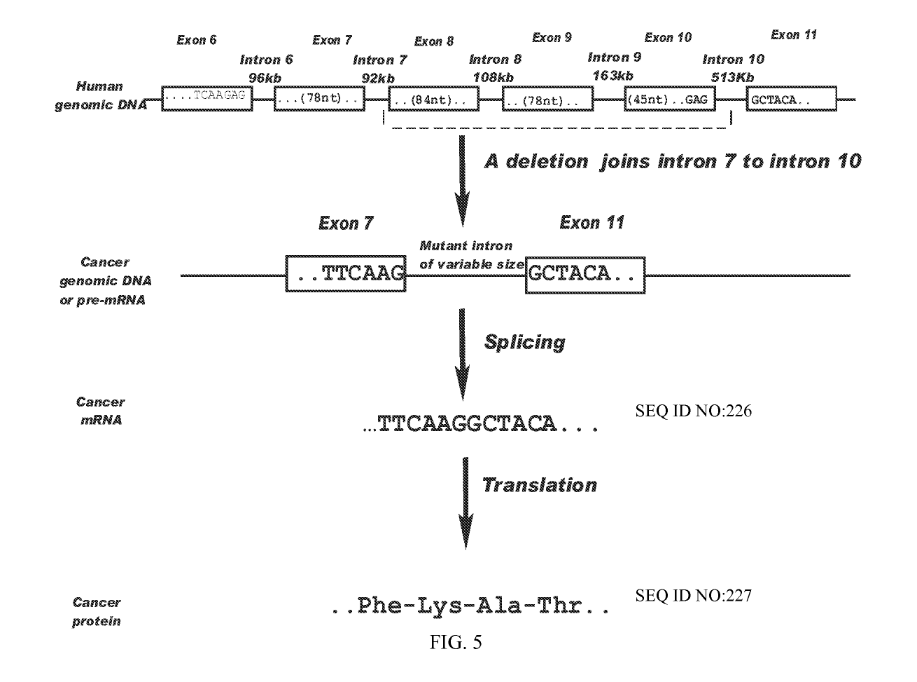

FIG. 6: The cancer mRNA connects the splice donor site of exon 8 with the splice acceptor site of exon 11. The RNA-to-protein translation joins GAG (Glu) CTG (Leu) of exon 8 to GCT (Ala) ACA (Thr) of exon 11.

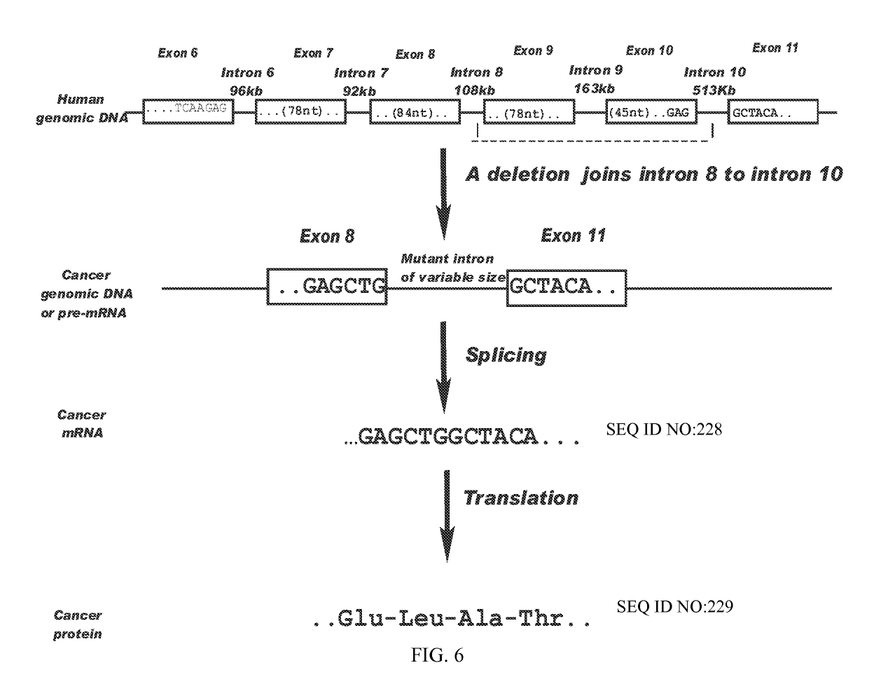

FIG. 7: The cancer mRNA connects the splice donor site in exon 8 with the splice acceptor site in exon 10. The RNA-to-protein translation joins GAG (Glu) CTG (Leu) of exon 8 to GGT (Gly) TTA (Leu) of exon 10.

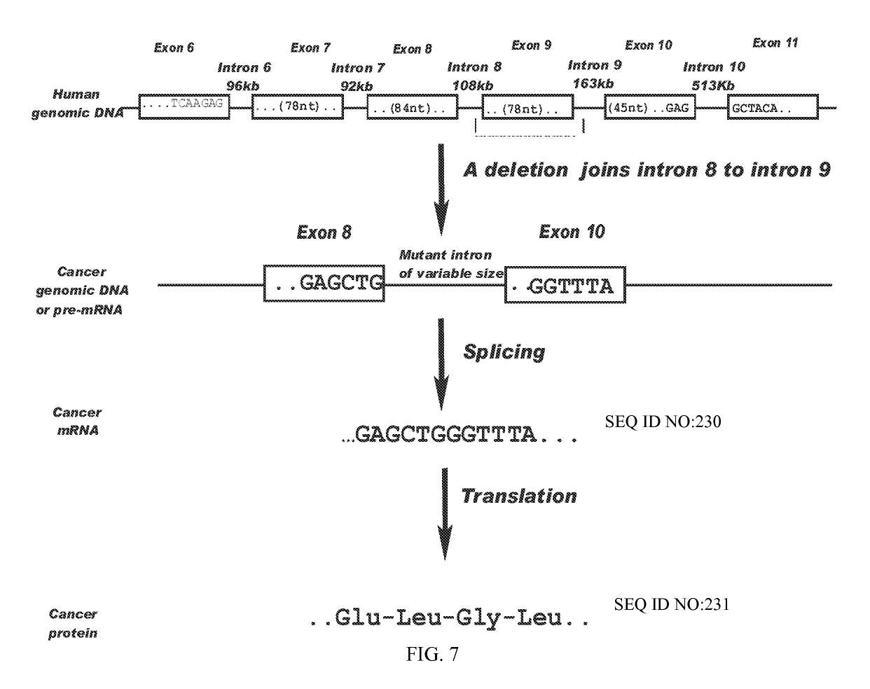

FIG. 8: The cancer mRNA connects the splice donor site in exon 9 with the splice acceptor site in exon 11. The RNA-to-protein translation joins TAT (Tyr) GAT (Asp) of exon 9 to GCT (Ala) ACA (Thr) of exon 11.

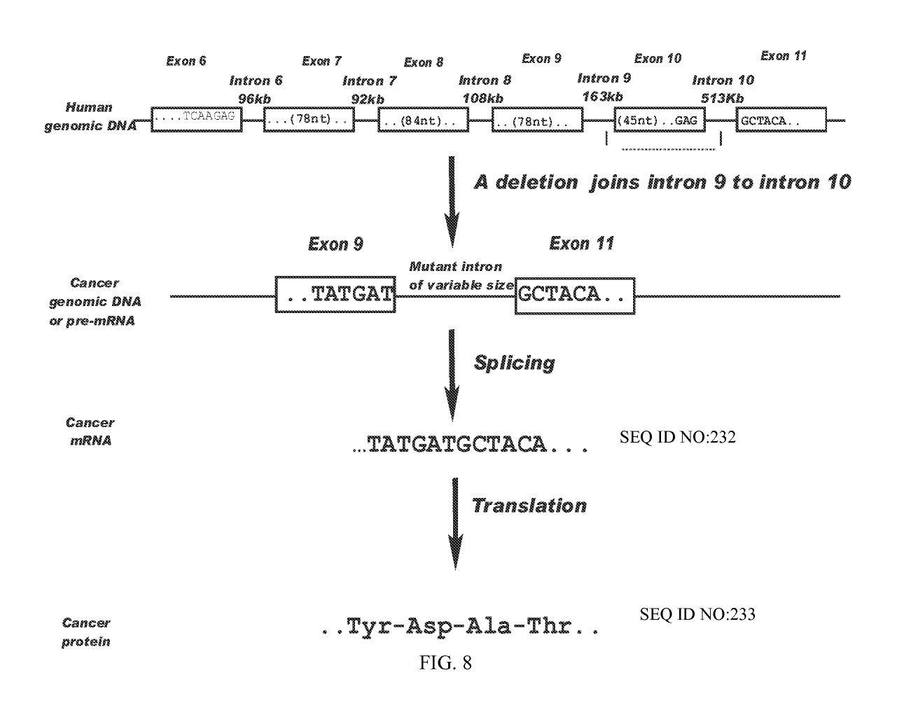

FIG. 9: The cancer mRNA connects the splice donor site in exon 3 with the splice acceptor site in exon 8. The RNA-to-protein translation joins AGT (Ser) TTG (Leu) of exon 3 to GAT (Asp) ATA (Ile) of exon 8.

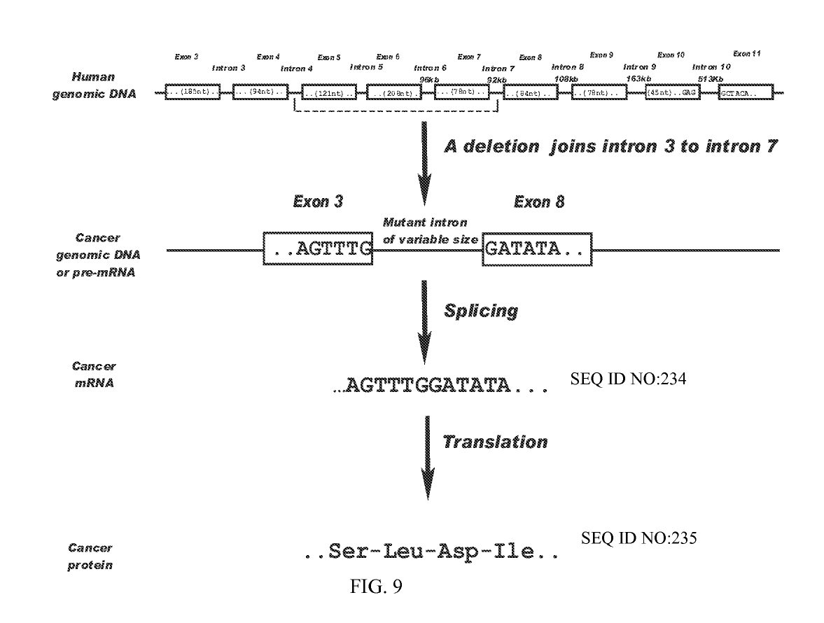

FIG. 10: The cancer mRNA connects the splice donor site in exon 3 with the splice acceptor site in exon 7. The RNA-to-protein translation joins AGT (Ser) TTG (Leu) of exon 3 to AGT (Ser) GCA (Ala) of exon 7.

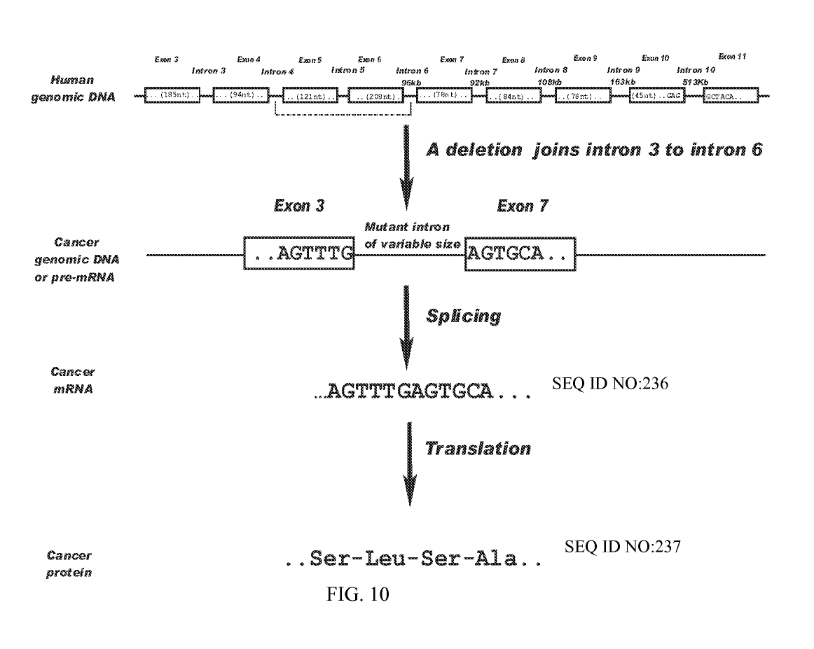

FIG. 11: The cancer mRNA connects the splice donor site in exon 3 with the splice acceptor site in exon 9. The RNA-to-protein translation joins AGT (Ser) TTG (Leu) of exon 3 to GGA (Gly) AAA (Lys) of exon 9.

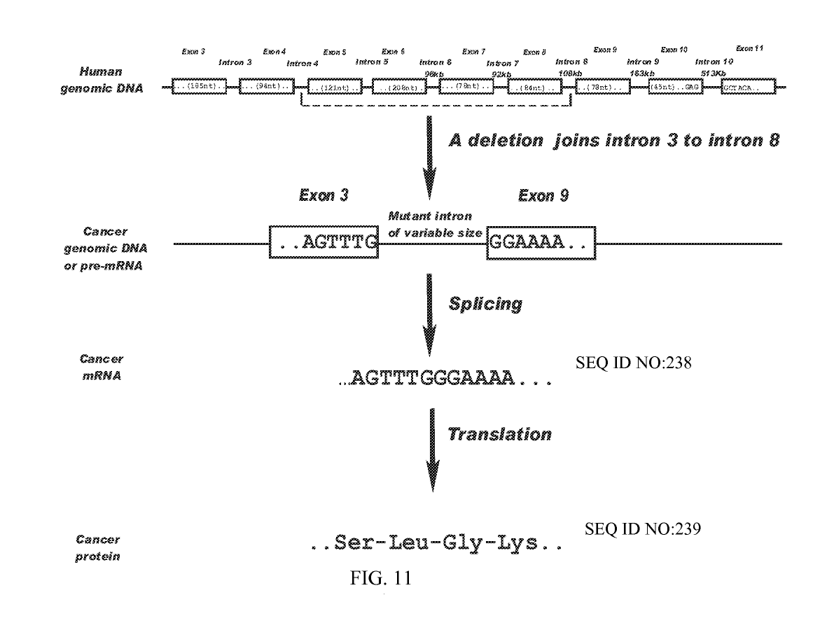

FIG. 12: The cancer mRNA connects the splice donor site in exon 3 with the splice acceptor site in exon 10. The RNA-to-protein translation joins AGT (Ser) TTG (Leu) of exon 3 to GGT (Gly) TTA (Leu) of exon 10.

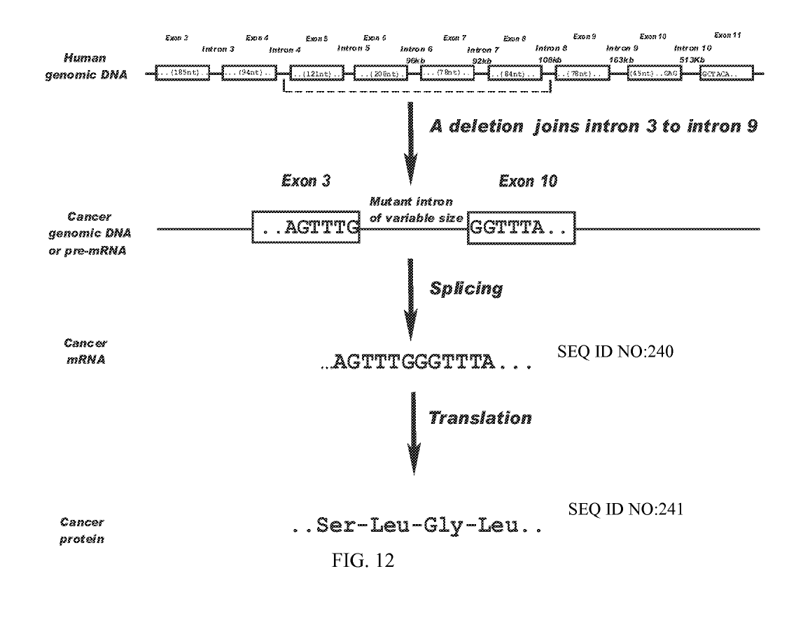

FIG. 13: The cancer mRNA connects the splice donor site in exon 3 with the splice acceptor site in exon 11. The RNA-to-protein translation joins AGT (Ser) TTG (Leu) of exon 3 to GCT (Ala) ACA (Thr) of exon 11.

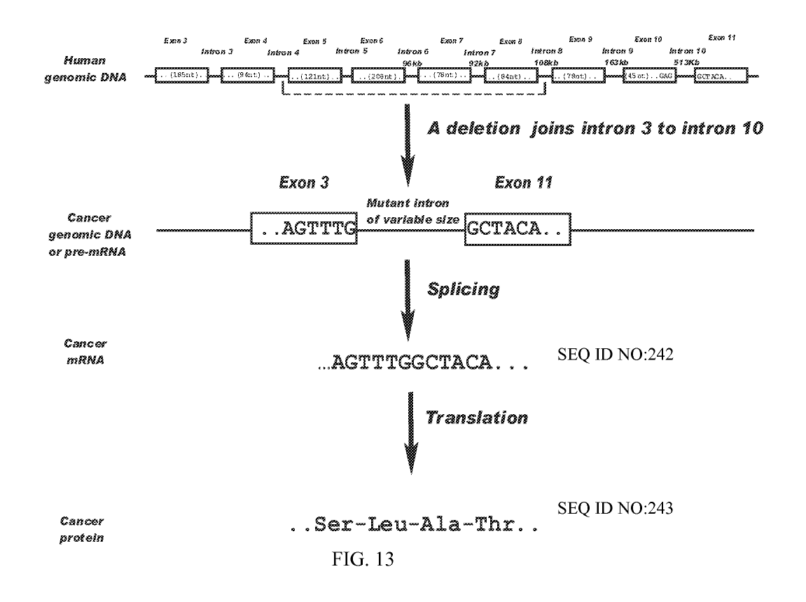

FIG. 14: The cancer mRNA connects the splice donor site in exon 6 with the splice acceptor site in exon 8. The RNA-to-protein translation joins CAA (Gln) GAG (Glu) of exon 6 to GAT (Asp) ATA (Ile) of exon 8.

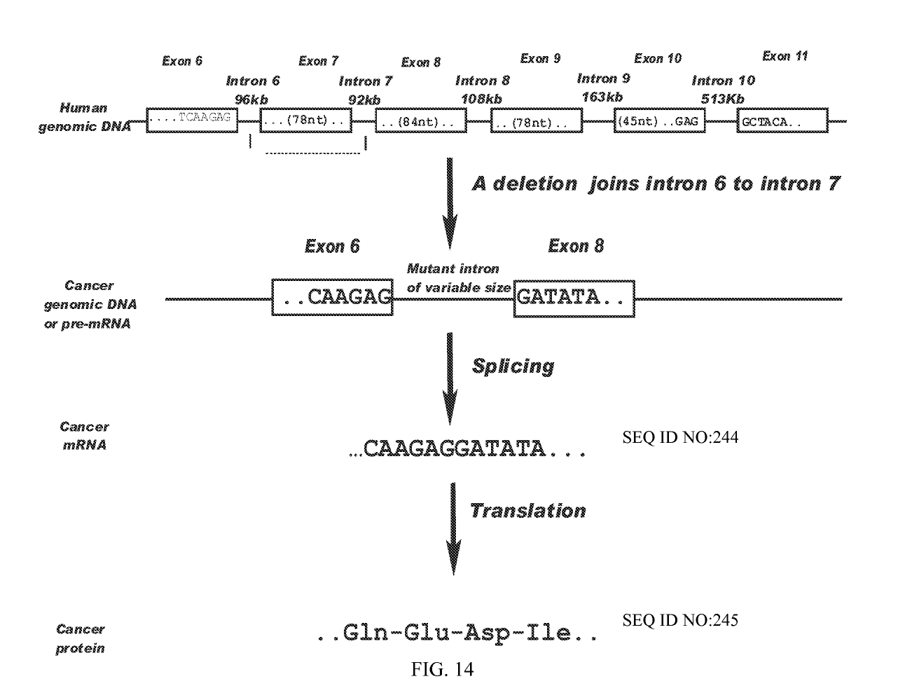

FIG. 15: The cancer mRNA connects the splice donor site in exon 7 with the splice acceptor site in exon 9. The RNA-to-protein translation joins TTC (Phe) AAG (Lys) of exon 7 to GGA (Gly) AAA (Lys) of exon 9.

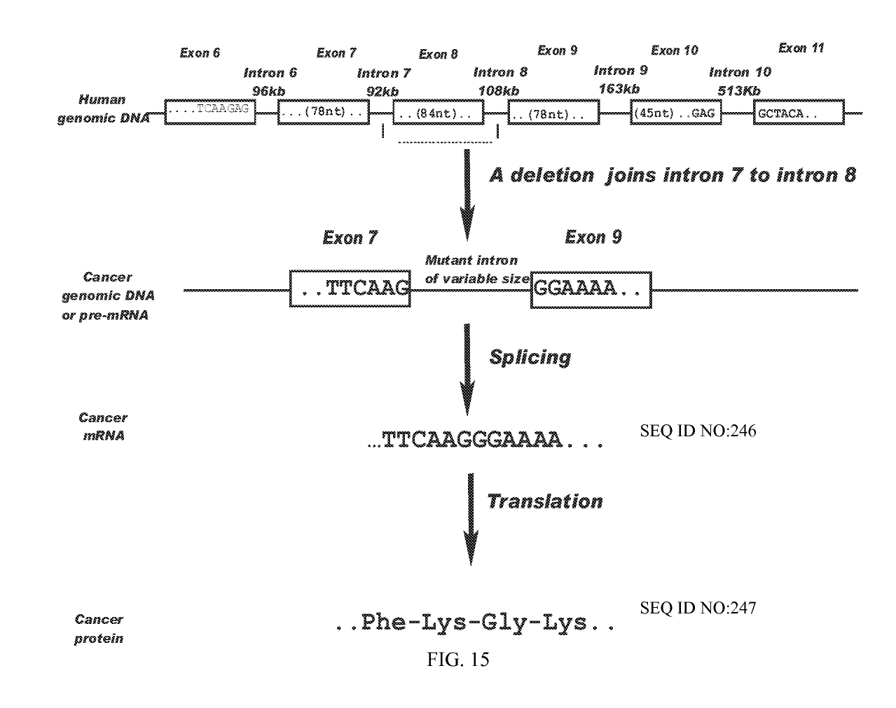

FIG. 16. (A) The genomic structure of FAM190A, NM_{—}001145065. Each box represents an exon, numbers above indicate their nucleotide lengths. Grey indicates the UTR; white and colors, the exons having nucleotide lengths not divisible and divisible by 3, respectively. (B) A magnification of exons from 3 to 11. (C) Top to bottom: structural types of the intragenic deletions, percentage of samples (combined panels) affected, percentage of homozygous deletions at the genomic level, type of rearranged transcripts, and predicted amino acidic sequences at the transcript rearrangement joints. *cDNA not examined. **Lack of a PCR product in cDNA from exons 6 to 11. ***Predicted new antigen at the joint between normally non-contiguous exons.

FIG. 17: Electropherograms showing a wild-type sequence and four of the 13 different rearrangements observed. For each cell line the 28 bp sequence of the anomalous fusion transcript is depicted. The two boxes on the top of each diagram represent the exon at the joint. Colors are as in FIG. 16.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

As described herein, the present inventors identified a novel intragenic rearrangement in several cancer xenografts and cell lines that creates an in-frame deletion in the mRNA of KIAA1680, a hypothetical gene recently renamed FAM190A (family with sequence similarity 190, member A). In the mRNA of each sample having the rearrangement, one of nine in-frame structures was observed: a deletion that removes exons 7 and 8, resulting in a mRNA where exon 6 is spliced to exon 9, a deletion that removes exon 7, 8 and 9, resulting in a mRNA where exon 6 is spliced to exon 10, a deletion that removes exons 7, 8, 9 and 10, resulting in a mRNA where exon 6 is spliced to exon 11, a deletion that removes exons 8 and 9, resulting in a mRNA where exon 7 is spliced to exon 10, a deletion that removes exons 8, 9 and 10, resulting in a mRNA where exon 7 is spliced to exon 11, a deletion that removes exons 9 and 10, resulting in a mRNA where exon 8 is spliced to exon 11, a deletion that removes exon 9 only, resulting in a transcript where exon 8 is spliced to exon 10, a deletion that removes exon 10 only, resulting in a mRNA where exon 9 is spliced to exon 11, and a deletion that removes exons 4, 5, 6, and 7, resulting in a mRNA where exon 3 is spliced to exon 8.

The cause of the rearranged mRNA species is mixed. In some tumors, a homozygous deletion of genomic DNA had removed one of the involved exons, which was consequently absent in the mRNA. In most tumors, a genomic cause was undetermined, and it was possible that the deletion occurred during processing of the pre-mRNA. In all instances, one can postulate that the pre-mRNA contains the deletion irrespective of a genomic or post-genomic defect.

### I. Definitions

The terms “nucleic acid,” “nucleic acid molecule,” “polynucleotide” or “polynucleotide molecule” are used interchangeably herein and refer to a polymeric form of nucleotides of any length, either ribonucleotides and/or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded nucleic acid product of chemical synthesis either by synthesizing the complementary strand and annealing the strands under appropriate conditions, or by synthesizing the complementary strand de novo using a DNA polymerase with an appropriate primer.

The following are non-limiting examples of nucleic acids: a gene or gene fragment, exons, introns, mRNA, tRNA, rRNA, ribozymes, cDNA, recombinant nucleic acids, branched nucleic acids, plasmids, vectors, isolated DNA of any sequence, isolated RNA of any sequence, nucleic acid probes, and primers. A nucleic acid may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid may be further modified after polymerization, such as by conjugation with a labeling component. Other types of modifications included in this definition are caps, substitution of one or more of the naturally occurring nucleotides with an analog, and introduction of means for attaching the nucleic acid to proteins, metal ions, labeling components, other nucleic acids, or a solid support.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

By “isolated nucleic acid (or polynucleotide) molecule” is meant a nucleic acid (e.g., a DNA, RNA, or analog thereof) that is free of the genes which, in the naturally occurring genome of the organism from which the nucleic acid molecule of the present invention is derived, flank the gene. The term therefore includes, for example, a recombinant DNA that is incorporated into a vector, into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

As used herein, the term “operably linked” means that nucleic acid sequences or proteins are operably linked when placed into a functional relationship with another nucleic acid sequence or protein. For example, a promoter sequence is operably linked to a coding sequence if the promoter promotes transcription of the coding sequence. As a further example, a repressor protein and a nucleic acid sequence are operably linked if the repressor protein binds to the nucleic acid sequence. Additionally, a protein may be operably linked to a first and a second nucleic acid sequence if the protein binds to the first nucleic acid sequence and so influences transcription of the second, separate nucleic acid sequence. Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins—transcription factors—or proteins which include transcriptional activator domains) are bound to the regulatory sequence or sequences.

The terms “amino acid” and “amino acid molecule” refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine.

An “amino acid analog” refers to a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain). The term “amino acid mimetic” refers to chemical compounds that have a structure that is different from the general chemical structure of an amino acid, but that function in a manner similar to a naturally occurring amino acid Amino acid analogs may have modified R groups (for example, norleucine) or modified peptide backbones, but retain the same basic chemical structure as a naturally occurring amino acid. In one embodiment, an amino acid analog is a D-amino acid, a beta-amino acid, or an N-methyl amino acid.

Amino acids and analogs are well known in the art Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

An “expression vector” or “vector” is a nucleic acid construct, generated recombinantly or synthetically, bearing a series of specified nucleic acid elements that enable transcription of a particular gene in a host cell. A vector is typically designed for transduction/transfection of one or more cell types. Typically, gene expression is placed under the control of certain regulatory elements, including constitutive or inducible promoters, tissue-preferred regulatory elements, and enhancers. In certain embodiments, an expression vector comprises nucleic acid molecule that encodes a mutant FAM190A protein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell. In certain embodiments, a “host cell” or “transformed cell” refers to a cell into which (or into an ancestor of which) has been introduced, by means of recombinant DNA techniques, a polynucleotide molecule encoding a mutant FAM190A protein of the present invention.

By “fragment” is meant a portion (e.g., at least about 5, 10, 25, 50, 100, 125, 150, 200, 250, 300, 350, 400, or 500 amino acids or nucleic acids) of a protein or nucleic acid molecule that is substantially identical to a reference protein or nucleic acid and retains at least one biological activity of the reference. In some embodiments the portion retains at least 50%, 75%, or 80%, or more preferably 90%, 95%, or even 99% of the biological activity of the reference protein or nucleic acid described herein.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “modulation” is meant a change (increase or decrease) in the expression level or biological activity of a gene or polypeptide as detected by standard methods known in the art. As used herein, modulation includes at least about 10% change, 25%, 40%, 50% or a greater change in expression levels or biological activity (e.g., about 75%, 85%, 95% or more).

By “recombinant” is meant the product of genetic engineering or chemical synthesis. By “positioned for expression” is meant that the polynucleotide of the present invention (e.g., a nucleic acid molecule) is positioned adjacent to a nucleic acid sequence that directs transcription and translation of the sequence (i.e., facilitates the production of, for example, a recombinant protein of the present invention, or an RNA molecule).

By “siRNA” is meant a double stranded RNA. Optimally, an siRNA is about 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to down regulate mRNA levels or promoter activity.

By “specifically binds” is meant a molecule (e.g., peptide, polynucleotide) that recognizes and binds a protein or nucleic acid molecule of the present invention, but which does not substantially recognize and bind other molecules in a sample, for example, a biological sample, which naturally includes a protein of the present invention. In certain embodiments, an antibody to a mutant FAM190A protein specifically binds such protein and does not substantially recognize and bind other molecules. In specific embodiments, such an antibody does not specifically bind wild type FAM190A protein. In other embodiments, such an antibody does not specifically bind other mutant FAM190A proteins. In particular embodiments, antibodies recognize the a portion of the mutant FAM190A protein that comprises the non-natural peptide sequence at the fusion joint (see, e.g., Table 1).

### II. FAM190A Polynucleotides

In general, the present invention features the use of nucleic acid sequences that encode a mutant FAM190A or biologically active fragment thereof. Also included in the methods of the present invention are nucleic acid molecules containing at least one strand that hybridizes with a FAM190A nucleic acid sequence (e.g., inhibitory nucleic acid molecules that reduce FAM190A expression, such as a dsRNA, siRNA, shRNA, or antisense oligonucleotides, microRNA, ribozymes, aptamers, monoclonal antibodies or other). An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### III. FAM190A Polypeptides

The present invention involves the production of mutant FAM190A proteins.

In one embodiment, FAM190As are expressed in host cells. In general, FAM190A proteins, variants, and fragments thereof are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the present invention. A polypeptide of the present invention may be produced in a prokaryotic host (e.g., E. coli) or in a eukaryotic host (e.g., Sacchamyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al.; expression vehicles may be chosen from those provided, e.g., in Cloning Vectors: A Laboratory Manual (P. H. Pouwels et al., 1985, Supp. 1987).

A variety of expression systems exist for the production of the FAM190A polypeptides of the present invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione 5-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3× may be cleaved with factor Xa.

Once the FAM190A mutant polypeptide of the present invention is expressed, it is isolated, e.g., using affinity chromatography. In one example, an antibody (e.g., produced as described herein) raised against a FAM190A mutant polypeptide of the present invention may be attached to a column and used to isolate the polypeptide. Lysis and fractionation of polypeptide-harboring cells prior to affinity chromatography may be performed by standard methods (see, e.g., Ausubel et al., supra).

Once isolated, the FAM190A mutant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the present invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful FAM190A mutant peptide fragments or analogs.

### IV. Therapeutic Methods

The present invention provides methods of treating a disease characterized by the presence of a FAM190A mutant protein described herein. In particular embodiments, the present invention provides methods for treating cancer including, but not limited to, breast, stomach, pancreas, colon, lung, kidney, and bladder. More specifically, the present invention can be useful in treating gastric adenocarcinoma, lymphoma, colorectal adenocarcinoma, and epithelioid carcinoma. The foregoing is not exhaustive; rather, the present invention is useful in treating any cancer in which a FAM190A mutant protein described herein is implicated.

The methods comprise administering a therapeutically effective amount of a pharmaceutical composition comprising an agent described herein (e.g., an agent that decreases FAM190A mutant protein expression or biological activity) to a subject (e.g., a mammal such as a human). Thus, in one embodiment, the present invention features a method of treating a subject suffering from or susceptible to a disease or disorder or symptom thereof characterized by the presence of a FAM190A mutant protein described herein. The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g., opinion) or objective (e.g., measurable by a test or diagnostic method).

The therapeutic methods of the present invention, which include prophylactic treatment, in general comprise administration of a therapeutically effective amount of the agents herein, such as a compound to a subject (e.g., animal, human) in need thereof, including a mammal, particularly a human. Such treatment will be suitably administered to subjects, particularly humans, suffering from, having, susceptible to, or at risk for a FAM190A-mediated disease like cancer. Determination of those subjects “at risk” can be made by any objective or subjective determination by a diagnostic test or opinion of a subject or health care provider (e.g., genetic test, enzyme or protein marker, marker (as defined herein), family history, and the like). The compounds herein may be also used in the treatment of any other disorders characterized by the presence of the FAM190A mutant proteins described herein or in which a FAM190A mutant protein described herein may be implicated.

### V. FAM190A Polynucleotide Therapy

Polynucleotide therapy featuring a polynucleotide encoding an inhibitory nucleic acid molecules that reduce mutant FAM190A expression (e.g., a dsRNA, siRNA, shRNA, or antisense oligonucleotides, (microRNA, ribozymes, aptamers, monoclonal antibodies or other) are therapeutic approaches for treating cancer. Such nucleic acid molecules can be delivered to cells of a subject or patient. The nucleic acid molecules must be delivered to the cells of a subject or patient in a form in which they can be taken up so that therapeutically effective levels of the inhibitory nucleic acid molecule can be produced.

Transducing viral (e.g., retroviral (lentiviral), adenoviral, and adeno-associated viral, herpes viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. 94:10319, 1997). For example, a polynucleotide encoding an inhibitory nucleic acid can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a FAM190A polynucleotide systemically.

Non-viral approaches can also be employed for the introduction of a therapeutic to a cell of a patient diagnosed as having cancer. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine. Administration should be sufficient to modulate expression of the mutant FAM190A protein and thus, cancer.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), Chicken Beta Actin (CBA) or metallothionein promoters). Promiscuous, ubiquitous or tissue/cell specific promoters are all useful in the methods of the present invention. The use of such promoters is routine. In other embodiments, promoters encompassed by the present invention are regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

A. Ribozymes

Catalytic RNA molecules or ribozymes that include an antisense mutant FAM190A sequence of the present invention can be used to inhibit expression of a mutant FAM190A nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the present invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the present invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

B. siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39. 2002).

Given the sequence of a target gene, siRNAs may be designed to inactivate that gene. Such siRNAs, for example, could be administered directly to an affected tissue, or administered systemically. The nucleic acid sequence of a FAM190A gene can be used to design small interfering RNAs (siRNAs). The 21 to 25 nucleotide siRNAs may be used, for example, as therapeutics to treat a FAM190A-mediated disease like cancer.

The inhibitory nucleic acid molecules of the present invention may be employed as double-stranded RNAs for RNA interference (RNAi)-mediated knock-down of mutant FAM190A expression. In one embodiment, mutant FAM190A expression is reduced in a cancer cell that expresses the mutant FAM190A protein. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the present invention, double stranded RNA (dsRNA) molecule is made that includes between eight and nineteen consecutive nucleobases of a nucleobase oligomer of the present invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

C. shRNAs

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

D. Aptamers

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. Desirably, the aptamers are small, approximately 15 KD. The aptamers are isolated from libraries consisting of some 10¹⁴-10¹⁵ random oligonucleotide sequences by a procedure termed SELEX (systematic evolution of ligands by exponential enrichment). See Tuerk et al., Science, 249:505-510, 1990; Green et al., Methods Enzymology. 75-86, 1991; Gold et al., Annu. Rev. Biochem., 64: 763-797, 1995; Uphoff et al., Curr. Opin. Struct. Biol., 6: 281-288, 1996. Methods of generating aptamers are known in the art and are described, for example, in U.S. Pat. Nos. 6,344,318, 6,331,398, 6,110,900, 5,817,785, 5,756,291, 5,696,249, 5,670,637, 5,637,461, 5,595,877, 5,527,894, 5,496,938, 5,475,096, 5,270,163, and in U.S. Patent Application Publication Nos. 20040241731, 20030198989, 20030157487, and 20020172962.

An aptamer of the present invention is capable of binding with specificity to a mutant FAM190A expressed by a cell of interest. “Binding with specificity” means that non-FAM190As or non-mutant FAM190As are either not specifically bound by the aptamer or are only poorly bound by the aptamer. In general, aptamers typically have binding constants in the picomolar range. Particularly useful in the methods of the present invention are aptamers having apparent dissociation constants of 1, 10, 15, 25, 50, 75, or 100 nM. In one embodiment, the present invention features a pharmaceutical composition that contains two or more aptamers, each of which recognizes a different mutant FAM190A.

In one embodiment, a mutant FAM190A is the molecular target of the aptamer. Because aptamers can act as direct antagonists of the biological function of proteins, aptamers that target a mutant FAM190A can be used to treat diseases characterized by the expression of mutant FAM190A, like cancer. The therapeutic benefit of such aptamers derives primarily from the biological antagonism caused by aptamer binding.

The present invention encompasses stabilized aptamers having modifications that protect against 3′ and 5′ exonucleases as well as endonucleases. Such modifications desirably maintain target affinity while increasing aptamer stability in vivo. In various embodiments, aptamers of the present invention include chemical substitutions at the ribose and/or phosphate and/or base positions of a given nucleobase sequence. For example, aptamers of the present invention include chemical modifications at the 2′ position of the ribose moiety, circularization of the aptamer, 3′ capping and “spiegelmer” technology. Such modifications are known in the art and are described herein. Aptamers having A and G nucleotides sequentially replaced with their 2′-OCH3 modified counterparts are particularly useful in the methods of the present invention. Such modifications are typically well tolerated in terms of retaining aptamer affinity and specificity. In various embodiments, aptamers include at least 10%, 25%, 50%, or 75% modified nucleotides. In other embodiments, as many as 80-90% of the aptatmer's nucleotides contain stabilizing substitutions. In other embodiments, 2′-OMe aptamers are synthesized. Such aptamers are desirable because they are inexpensive to synthesize and natural polymerases do not accept 2′-OMe nucleotide triphosphates as substrates so that 2′-OMe nucleotides cannot be recycled into host DNA. A fully 2′-O-methyl aptamer, named ARC245, was reported to be so stable that degradation could not be detected after 96 hours in plasma at 37° C. or after autoclaving at 125° C. Using methods, described herein, aptamers will be selected for reduced size and increased stability. In one embodiment, aptamers having 2′-F and 2′-OCH₃ modifications are used to generate nuclease resistant aptamers. Other modifications that stabilize aptamers are known in the art and are described, for example, in U.S. Pat. No. 5,580,737; and in U.S. Patent Application Publication Nos. 20050037394, 20040253679, 20040197804, and 20040180360.

E. Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells. See, e.g., U.S. Pat. Nos. 5,656,611; 5,753,613; 5,785,992; 6,120,798; 6,221,959; 6,346,613; and 6,353,055, each of which is hereby incorporated by reference.

### VI. Antibodies to Mutant FAM190A Proteins

In another approach, the present invention features methods for treating cancer, for example, by reducing the biological activity of a FAM190A mutant protein. Methods for reducing the biological activity of a FAM190A include administering to a subject in need thereof an antibody that specifically binds and disrupts the biological activity of a FAM190A mutant described herein. Antibodies are well known to those of ordinary skill in the science of immunology. As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the present invention comprise whole native anti-bodies, bispecific antibodies; chimeric antibodies; Fab, Fab′, single chain V region fragments (scFv) and fusion polypeptides.

In one embodiment, an antibody that binds a FAM190A mutant is monoclonal. Alternatively, the anti-mutant FAM190A antibody is a polyclonal antibody. The preparation and use of polyclonal antibodies are also known the skilled artisan. The present invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(aba)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab.alpha. fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing mutant FAM190As, or immunogenic fragments thereof, as an immunogen or immunogenic agent. One method of obtaining antibodies is to immunize suitable host animals with an immunogenic agent and to follow standard procedures for polyclonal or monoclonal anti-body production. The immunogenic agent will facilitate presentation of the immunogenic agent on the cell surface. Immunization of a suitable host can be carried out in a number of ways. Nucleic acid sequences encoding a FAM190A mutant, or immunogenic fragments thereof, can be provided to the host in a delivery vehicle that is taken up by immune cells of the host. The cells will in turn express the polypeptide, thereby generating an immunogenic response in the host. Alternatively, nucleic acid sequences encoding a FAM190A mutant or immunogenic fragments thereof, can be expressed in cells in vitro, followed by isolation of the polypeptide and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the present invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567; 5,530,101; 5,225,539; 5,585,089; 5,693,762; and 5,859,205.

### VII. Pharmaceutical Compositions

In several embodiments, the present invention contemplates pharmaceutical preparations comprising an aptamer that binds a mutant FAM190A, an antibody that specifically binds and neutralizes a mutant FAM190A protein, or a mutant FAM190A inhibitory nucleic acid molecule (e.g., a polynucleotide that hybridizes to and interferes with the expression of a mutant FAM190A polynucleotide), together with a pharmaceutically acceptable carrier. Polynucleotides of the present invention may be administered as part of a pharmaceutical composition. The compositions should be sterile and contain a therapeutically effective amount of the polypeptides or nucleic acid molecules in a unit of weight or volume suitable for administration to a subject.

These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous FAM190A polynucleotide solution, such as an aqueous solution of FAM190A polynucleotide or polypeptide, and the resulting mixture can then be lyophilized. The infusion solution can be prepared by reconstituting the lyophilized material using sterile Water-for-Injection (WFI).

The FAM190A polynucleotide or polypeptide may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a neoplastic disease or disorder, an effective amount is sufficient to stabilize, slow, or reduce the proliferation of the neoplasm. Generally, doses of active polynucleotide or polypeptide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the FAM190A polynucleotide or polypeptide compositions of the present invention.

A variety of administration routes are available. The methods of the present invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

Nanoparticles are a colloidal carrier system that has been shown to improve the efficacy of the encapsulated drug by prolonging the serum half-life. Polyalkylcyano-acrylates (PACAs) nanoparticles are a polymer colloidal drug delivery system that is in clinical development, as described by Stella et al., J. Pharm. Sci., 2000. 89: p. 1452-1464; Brigger et al., Int. J. Pharm., 2001.214: p. 3742; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly(acetic acid) (PLA) and poly (lactic-o-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) orpoly (lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the present invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the present invention include PEG-PLGA polymers.

Compositions of the present invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best-treatment regimens for using compounds of the present invention to treat a disease characterized by, for example, mutant FAM190A can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Where a composition of the present invention is used dosages of 1 mg, 2 mg, 3 mg, 5 mg, 7 mg, 10 mg, 15 mg, 20 mg, or 25 mg can be used per day. Of course, this dosage amount may be adjusted upward or downward, as is routinely done in such treatment protocols, depending on the results of the initial clinical trials and the needs of a particular patient. In various embodiments, compositions of the present invention are administered directly to a tissue or organ of interest by direct injection of a protein or inhibitory nucleic acid molecule described herein or by injection of a vector, such as a viral vector encoding a protein or inhibitory nucleic acid molecule of interest. In one approach, a therapeutic composition is administered in or near the target tissue.

### VIII. Screening Assays

Compounds that modulate the expression or activity of a mutant FAM190A protein, variant, or fragment thereof are useful in the methods of the present invention for the treatment or prevention of a disease or disorder characterized by the expression of the mutant FAM190A such as cancer. Any number of methods are available for carrying out screening assays to identify such compounds. In one approach, candidate compounds are identified that specifically bind to and alter the activity of a polypeptide of the present invention (e.g., a mutant FAM190A activity associated with cancer). Methods of assaying biological activities are known in the art and are described herein. The efficacy of such a candidate compound is dependent upon its ability to interact with a mutant FAM190A, variant, or fragment. Such an interaction can be readily assayed using any number of standard binding techniques and functional assays (e.g., those described in Ausubel et al). For example, a candidate compound may be tested in vitro for interaction and binding with a polypeptide of the present invention and its ability to affect cell proliferation. Standard methods for perturbing or reducing mutant FAM190A expression include interfering with FAM190A expression using RNAi, or microinjecting a mutant FAM190A-expressing cell with an antibody or aptamer that binds mutant FAM190A and interferes with its function.

Potential antagonists of a mutant FAM190A include organic molecules, peptides, peptide mimetics, polypeptides, nucleic acid molecules (e.g., double-stranded RNAs, siRNAs, antisense polynucleotides, aptamers), and antibodies that bind to a nucleic acid sequence or polypeptide of the present invention and thereby inhibit or extinguish its activity. Potential antagonists also include small molecules that bind to the mutant FAM190A thereby preventing binding to cellular molecules with which the FAM190A normally interacts, such that the biological activity of the mutant FAM190A is reduced or inhibited. Small molecules of the present invention preferably have a molecular weight below 2,000 daltons, more preferably between 300 and 1,000 daltons, and most preferably between 400 and 700 daltons. It is preferred that these small molecules are organic molecules.

In one particular example, a candidate compound that binds to a mutant FAM190A protein, variant, or fragment thereof may be identified using a chromatography-based technique. For example, a polypeptide of the present invention may be purified by standard techniques from cells engineered to express the polypeptide (e.g., those described above) and may be immobilized on a column. A solution of candidate compounds is then passed through the column, and a compound specific for the mutant FAM190A is identified on the basis of its ability to bind to the mutant FAM190A and be immobilized on the column. To isolate the compound, the column is washed to remove non-specifically bound molecules, and the compound of interest is then released from the column and collected.

Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to alter the biological activity of a mutant FAM190A.

In certain embodiments, compounds that are identified as binding to a polypeptide of the present invention with an affinity constant less than or equal to about 10 mM are considered particularly useful in the present invention. Alternatively, any in vivo protein interaction detection system, for example, any two-hybrid assay may be utilized to identify compounds that interact with a mutant FAM190A. Interacting compounds isolated by this method (or any other appropriate method) may, if desired, be further purified (e.g., by high performance liquid chromatography). Compounds isolated by any approach described herein may be used as therapeutics to treat cancer in a human patient.

In addition, compounds that inhibit the expression of a mutant FAM190A nucleic acid molecule whose expression is altered in a patient having cancer are also useful in the methods of the present invention. Any number of methods are available for carrying out screening assays to identify new candidate compounds that alter the expression of a mutant FAM190A nucleic acid molecule. In one working example, candidate compounds are added at varying concentrations to the culture medium of cultured cells expressing one of the nucleic acid sequences of the present invention. Gene expression is then measured, for example, by microarray analysis, Northern blot analysis, or RT-PCR, using any appropriate fragment prepared from the nucleic acid molecule as a hybridization probe. The level of gene expression in the presence of the candidate compound is compared to the level measured in a control culture medium lacking the candidate molecule. A compound that promotes an alteration in the expression of a mutant FAM190A gene, or a functional equivalent thereof, is considered useful in the present; such a molecule may be used, for example, as a therapeutic to treat cancer in a human patient.

In another approach, the effect of candidate compounds is measured at the level of polypeptide production to identify those that promote an alteration in a mutant FAM190A level. The level of mutant FAM190A can be assayed using any standard method. Standard immunological techniques include Western blotting or immunoprecipitation with an antibody specific for a mutant FAM190A. For example, immunoassays may be used to detect or monitor the expression of at least one of the mutant polypeptides of the present invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes a decrease in the expression or biological activity of the mutant polypeptide is considered particularly useful. Again, such a molecule may be used, for example, as a therapeutic to delay, ameliorate, or treat a FAM190A-mediated disease like cancer in a human patient.

In another embodiment, a nucleic acid described herein is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of a FAM190A-mediated disease like cancer. In one embodiment, the compound decreases the expression of the reporter.

Each of the DNA sequences referenced herein may also be used in the discovery and development of a therapeutic compound for the treatment of a FAM190A-mediated disease like cancer. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques.

The present invention also includes novel compounds identified by the above-described screening assays. Optionally, such compounds are characterized in one or more appropriate animal models to determine the efficacy of the compound for the treatment of a FAM190A-mediated disease like cancer. Desirably, characterization in an animal model can also be used to determine the toxicity, side effects, or mechanism of action of treatment with such a compound. Furthermore, novel compounds identified in any of the above-described screening assays may be used for the treatment of a disease in a subject. Such compounds are useful alone or in combination with other conventional therapies known in the art.

### IX. Test Compounds and Extracts

In general, compounds capable of inhibiting the growth or proliferation of a cancer by altering the expression or biological activity of a FAM190A mutant protein, variant, or fragment thereof are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and Pharma1Mar, U.S. (Cambridge, Mass.).

In one embodiment, test compounds of the present invention are present in any combinatorial library known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer. DrugDes. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

Those skilled in the field of drug discovery and development will understand that the precise source of a compound or test extract is not critical to the screening procedure(s) of the present invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of a FAM190A mutant protein, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, compounds shown to be useful agents for the treatment of a FAM190A-mediated disease like cancer are chemically modified according to methods known in the art.

### X. Kits or Pharmaceutical Systems

The present compositions may be assembled into kits or pharmaceutical systems for use in ameliorating cancer. Kits or pharmaceutical systems according to this aspect of the present invention comprise a carrier means, such as a box, carton, tube or the like, having in close confinement therein one or more container means, such as vials, tubes, ampules, bottles and the like. The kits or pharmaceutical systems of the present invention may also comprise associated instructions for using the agents of the present invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the present invention, and, as such, may be considered in making and practicing the present invention. Particularly useful techniques for particular embodiments will be discussed in the sections that follow.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Sample Collection. Seventy-two human tumor specimens, 39 cell lines and 33 xenografts, were studied. Twenty of the cell lines (AsPc1, BT-20, BT-474, CAPAN1, CAPAN2, CFPAC1, COLO357, DLD-1, HeLa, Hs578T, MCF7, MDA-MB-134, MDAMB-453, MiaPaCa2, Panc-1, P215, PL45, T470, RKO, HEK 293) were randomly chosen from those available to us; the other 19 (AGS, BC-1, BxPc3, COLO0205, H508, H727, HT-1376, H1581, H1975, H2126, H2228, KATO III, LNCa-Clone-FGC, LoVo, SW620, SW403, SW780, SW837, and SW1417) were selected for a having a known deletion affecting 4q22 (7). The cell lines were obtained from European Collection of Cell Cultures (ECACC) (COLO357, P215) and American Type Culture Collection (ATCC).

Thirty-three xenografted human cancers of different types were obtained from our described tissue banks (23) under an IRB-approval protocol. Of these samples, 5 were selected for a known deletion affecting 4q22 (PX19, PX19-2R, PX19-3, PX19-4, PX188) (24) and 28 were unselected.

A panel of cDNAs from 48 different human normal tissues, was obtained (TissueScan, OriGene).

Three mouse cell lines (CT-38, LLC, MEF-P3) and a panel of 36 normal samples representing 18 different tissues taken from newborn and adult mice were studied by RT-PCR. The organs included heart, stomach, kidney, liver, lung, brain cerebellum, brain brainstem, brain cortex, pancreas, thymus, spleen, salivary gland, adrenal gland, skin, colon and small intestine.

DNA and RNA Isolation. Total RNA was extracted from cell lines and tumors (Trizol, Invitrogen). Purification was performed using columns according to manufacturer's instructions (Rneasy, Qiagen). RNA quality was assessed by gel electrophoresis of ethidium-bound total RNA. RNA was treated with DNase I (Invitrogen) and retrotranscribed (SuperScript® III, Invitrogen) to form cDNA.

Genomic DNA was extracted according to manufacturer's instructions (QIAamp, Qiagen). DNA and RNA concentrations were determined using spectrometry (NanoDrop Technologies).

Primer Design, PCR, and Sequence Analysis. The primers were designed using Primer3 (http://frodo.wi.mit.edu/) and synthesized by Integrated DNA Technologies (IDT) (Table 5-7). Designed primers were aligned against the corresponding genome sequence using BLAT (http://genome.ucsc.edu/cgi-bin/hgBlat, assembly February 2009, GRCh37/hg19) to confirm specificity. Taq DNA polymerase was used for the PCR reactions.

PCR conditions were as follows: 94° C. for 4 min, 72° C. for 10 s, and then 40 cycles of 94° C. for 30 s, 55° C. for 30 s, 72° C. for 30 s. PCR products were separated on 1% agarose gel in lithium boric acid buffer (LB®, Faster Better Media LLC) (25) to determine presence and size, processed (QIAquick PCR Purification Kit, Qiagen) and analyzed by automated sequencing.

RACE PCR. 5′RACE PCR was performed (FirstChoice RLMRACE, Applied Biosystem) according to manufacturer's instructions.

### Example 1

### Structure of the FAM190A Coding Sequence

Partially overlapping homozygous genomic deletions (HDs) of 4q22.1 were found in a pancreatic cancer cell line, BxPc3 and in two of 60 xenografted pancreatic cancers, PX19 and PX188. A fourth homozygous deletion of the same region was reported in a lung cancer line, H2126 (12), and a somatic out-of-frame deletion of two exons is reported in multiple metastases of a single pancreatic cancer (13). In all five cases, the overlapping deleted region included the FAM190A gene.

In order to analyze the transcriptional pattern of FAM190A, overlapping primers for the human FAM190A transcript variant 1 were designed (see Materials and Methods and Table 3). A PCR-based screen of exons 2 to 11 was then performed on cDNAs synthesized from 72 cell lines and xenografted cancer of different types. This sample set comprised two panels: 48 unselected samples and 24 samples selected for having a known HD or small heterozygous deletions at 4q22.1 (Table 4) (7). The gene was expressed in most samples (92%, 66/72 cases). DNA fragments of unexpected size were sequenced. In the former panel eight types of rearranged transcripts and internal rearrangements were found in nearly 40% of the cases (39.6%, 19/48 cases). Among these affected samples, 84% had in-frame structures, 94% of which involved deletion of exon 9.

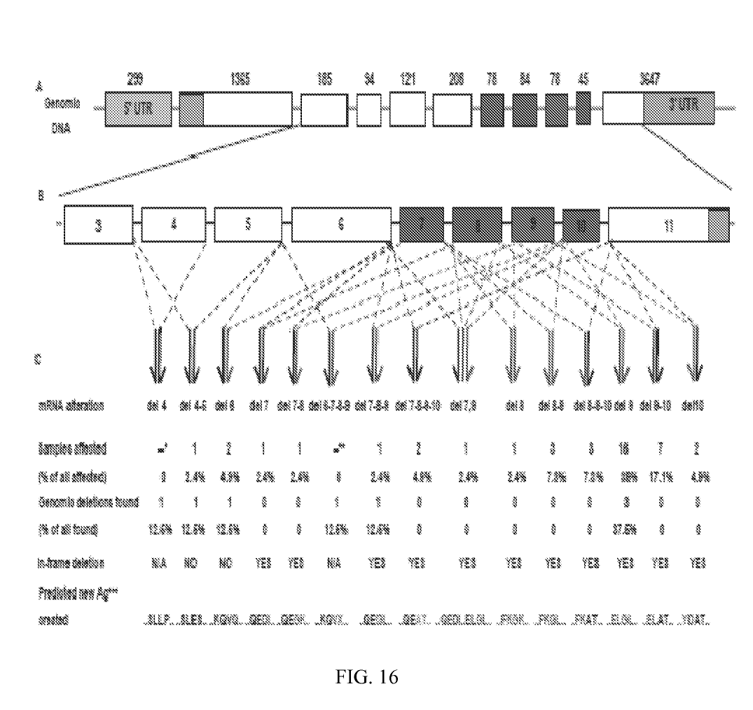

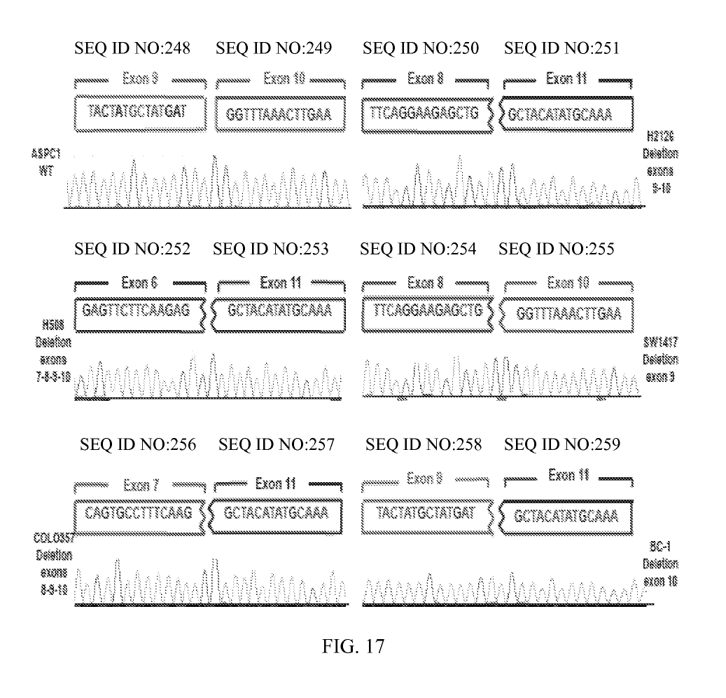

In the latter panel, nine types of rearrangements and 18 aberrant cases (75%) producing exclusively in-frame structures were found. In 89% of the cases, the deletion involved exon 9. For each case having a deleted FAM190A transcript, a co-existing spliced form of expected length (wild-type) and/or only a rearranged transcript were found. Among combined selected and unselected cases (FIG. 16), the changes appeared homozygous in 24 samples and heterozygous in 13. Overall, 13 aberrant structural transcript types were identified between exons 2 and 11: 11 represented different in-frame deletions (FIGS. 16 and 17); the remaining two caused shifting of the reading frame. Two cell lines and one xenograft (H1975, SW780, and MX7) had multiple rearranged spliced forms. Sequencing of the cDNA of AsPc1 revealed no subtle (point) mutation in the coding sequence. The sequencing of the cDNA of BxPc3 revealed (beside the known deletion of exon 9 and 10) the presence of a heterozygous nonsynonymous SNP at nucleotide 1144 resulting in a missense mutation at aminoacid 382. All rearrangements could be replicated in a PCR assay using different primers. In one patient, four independent parallel xenografts (PX19-1, -2, -3, and -4) had been derived from four locations in the resected primary tumor. All four had the identical exon deletion, indicating that the deletion had pre-existed within the patient's tumor prior to expansion as xenografts.

At the protein level, the in-frame deletions were predicted to form novel peptide sequences (Table 1). These are presumptive cancer-specific “neo-antigens”.

TABLE 1
Variations in the FAM190A coding region
transcripts.

| Deleted | | Predicted non-natural |
|---|---|---|
| | | peptide sequence at the |
| Exons | Consequence | fusion joint* |
| 4-5 | Not in | SSSSKMNSL~ESFPEINKGRX† |
| | frame | (SEQ ID NO: 54) |
| 6 | Not in | PEFPEPSK~QVQTX |
| | frame | (SEQ ID NO: 55) |
| 7 | In frame | LKMKRVLQE~DIMKDECSM |
| | | (SEQ ID NO: 56) |
| 7-8 | In frame | LKMKRVLQE~GKVRHLQKA‡ |
| | | (SEQ ID NO: 57) |
| 7-8-9 | In frame | LKMKRVLQE~GLNLKRLET |
| | | (SEQ ID NO: 58) |
| 7-8-9-10 | In frame | LKMKRVLQE~ATYRNRIVS |
| | | (SEQ ID NO: 59) |
| 7 and 9 | In frame | LKMKRVLQE~DIMKDECSMLKLQL |
| | | KEKDELISQLQEEL~GLNEKRLET§, \|\| |
| | | (SEQ ID NO: 60) |
| 8 | In frame | LTEEPVPFK~GKVRHLQKA |
| | | (SEQ ID NO: 61) |
| 8-9 | In frame | LTEEPVPFK~GLNEKRLET†† |
| | | (SEQ ID NO: 62) |
| 8-9-10 | In frame | LTEEPVPFK~ATYRNRIVS |
| | | (SEQ ID NO: 63) |
| 9 | In frame | LISQLQEEL~GLNEKRLET§ |
| | | (SEQ ID NO: 64) |
| 9-10 | In frame | LISQLQEEL~ATYRNRIVS** |
| | | (SEQ ID NO: 65) |
| 10 | In frame | TQTELLCYD~ATYRNRIVS |
| | | (SEQ ID NO: 66) |

Before the ~ sign are listed the last nine amino acid residues of the exon preceding the deletion.
After the ~ sign are listed the first nine amino acid residues of the exon following the deletion.
An underline indicates the amino acidic residues that would be created due to a frame-shift.
These two not-in-frame deletions would create a truncated predicted polypeptide.
†X, stop codon.
For HLA-A*0205, the cognate ligand structure -q-g-V--L matches del7-8‡ and the structure -L---L--L matches the joints produced by del7 and 9§ and del9§.
For HLA-A3, the ligand structure -V----I-K- matches del7 and 9||, the structure -L---L--Y- matches del9-10**, and the structure --F--L--K- matches del8-9†† [26]

The same PCR and sequencing analysis from exon 6 to exon 11 was conducted on 48 commercially available cDNAs from different normal human tissues. Reproducible expression of FAM190A transcripts, assessed by multiple independent replicates, was found in 37 samples, 33 of which had a wild-type FAM190A transcript exon structure. Four samples had an exon deletion observed once: in one case exon 9 was lost; in one exon 8; and in two, exon 7. Two samples had a cryptic intronic exon inserted, each observed once. Additional samples of the organs, however, did not confirm the observed deletion or insertion, and these were considered as unconfirmed alternative splice variants.

### Example 2

### Structure of FAM190A Transcripts in the 5′ Noncoding Region

5′RACE PCR was performed to determine: 1) the transcript variants expressed in our samples and 2) the structure of the FAM190A transcript in the 5′ non-coding region. In a pancreatic cancer cell line, AsPc1, variant 1 and a novel variant 3 having an alternative first exon were observed. Based on this knowledge, RT-PCR conducted on a second pancreatic cancer cell line, BxPc3, revealed in addition to variant 1, the presence of variant 3 and a novel variant 4 having an alternative first and second exon. Variant 2 was never observed by us in the samples analyzed. In all four variants, the apparent start codon ATG is at the position 91229395 (Table 2).

TABLE 2
5′ variations in the FAM190A transcript.

| | |
|---|---|
| 5′-most exon* | Additional 5′ exon |

| Variants | Start (bp) | End (bp) | Start | End |
|---|---|---|---|---|
| #1 | 91048684 | 91048982 | — | — |
| #2‡ | 91156182 | 91156494 | — | — |
| #3 | 91049688 | 91049813 | — | — |
| #4 | 91049688 | 91049813 | 91184749 | 91184873 |

*The genomic positions of the exons are expressed in base pairs according to build GRCh37/hg19.
^{†} The exon containing the starting codon ATG.
^{‡}Variant 2 as annotated in the USCS Genome Browser (http://genome.ucsc.edu/), unconfirmed by us.
Additional unconfirmed 5′ variants are reported in the Ensembl database (http://uswest.ensembl.org/index.html).

### Example 3

### Structure of the Genomic FAM190A

In order to rationalize the transcripts through their genomic structures, PCR analysis was performed at the intron/exon junctions of exons 4 through 10 of 45 genomic DNAs isolated from 37 cancer cell lines and eight pancreatic cancer xenografts. Among these samples, 23 had rearrangements of their transcripts. In nine, the transcript alterations were fully and/or partially explained by homozygous losses of genomic material, which encompassed only the exons deleted in the corresponding transcript. Mechanisms other than genomic deletion might underlie the aberrant transcripts in the 14 remaining cases, such as undiscovered intronic point mutations, small deletions affecting splicing signals, and heterozygous or compound heterozygous genomic deletions.

### Example 4

### Mouse FAM190A Gene (FAM190A)

Four mouse CFSs have been defined at the molecular level. Of these, Fra6C1 corresponds to the human FRA4F (6). The murine ortholog of the FAM190A gene, FAM190A, maps to mouse chromosome 6 and has two isoforms which differ in the 5′UTR but encode the same protein. The human and the mouse transcripts share 82% identity of the nucleotide coding sequence, suggesting that a deletion pattern similar to that observed in the human was possible. To test this possibility, an RT-PCR-based screening analysis was performed on two murine cancer cell lines (CT38, LLC) and one murine embryonic fibroblast line (MEF-P3) along with 36 different normal murine tissues. An amplified fragment was produced in 27 samples, which was of expected size, suggesting that no rearrangements were present. Widespread expression of FAM190A was observed in both newborn and adult murine tissues.

The contribution of rare and common fragile sites to genome rearrangements and diseases has long been studied. Perhaps owing to an unusual nucleotide composition and high structural flexibility, fragile sites have delayed replication in S phase, a characteristic that may lead to the formation of local replicative gaps and illegitimate chromosomal rearrangements, and result in fixed genomic deletions.

It is still not clear to what extent these play a role in cancer. Recurrent, low-frequency deletions that do not retain the reading frame can affect the coding exons of the FHIT and WWOX genes at the respective fragile sites, and intronic deletions not affecting the structure of the mature mRNA also are seen (14, 15). These patterns have lead to the controversy whether these genes may be either “driver” tumor-suppressor genes or instead reflect the uncovering of “passenger” random changes affecting fragile sites (16, 17, 18).

As disclosed herein, the present invention describe the finding of structural defects in the FAM190A transcript in 40% of human cancers and transformed cells. Evidence for widespread rearrangements affecting this region in multiple tumor types suggests that the mutant coding sequences identified might be among the most frequent mutations in human cancer. This high frequency is not readily explained by the mere coincidental location of FAM190A in a fragile region, for the FRA4F site spans about 10 megabase pairs, and the affected region evaluated here is less than 5% of that span. Nor does FAM190A have a deletion pattern in cancers similar to other altered genes evaluated at fragile sites, even if attention was restricted to the exons (or groups of contiguous exons) contained in these genes in which the nucleotide count is a perfect multiple of “3”. The remaining plausible possibility is that the FAM190A changes of cancers is selective, wherein certain particular deletions arising from random processes has become enriched due to providing a growth advantage during neoplastic progression. This selection appears to preferentially act upon gross rearrangements, for whole exomic and whole-genomic sequencing of human cancers (including ours) (19) has not found sub-exonic subtle mutations of this gene such as missense or nonsense mutations.

The deletions of FAM190A might, in theory, be recessive or dominant during tumorigenesis. Of the rearranged transcripts, 93% remained in-frame at the fusion (intragenic translocation) joint. Thirteen were apparently heterozygous, for a normal transcript coexisted with the mutant form. This suggests that the mutant protein products may retain, provide new (or gain a) function and they are dominant. Dominant mutant genes selected during oncogenesis are classified as oncogenes.

Some of the in-frame rearrangements of the transcript corresponded to the exons spanned by intragenic homozygous deletions of the genomic DNA. In other instances, a genomic basis was implied, for the prevalence rate of FAM190A transcript alterations was elevated in cancers pre-selected for known heterozygous and homozygous genomic DNA deletions in the neighborhood. In the remaining tumors having no exonic genomic deletions, an undiscovered intronic mutation (similar perhaps to the genomic intronic mutations of the CD22 gene in B-precursor leukemia proposed as causing exon 12 deletions in the transcripts) (20) or an epigenetic mechanism may be the underlying cause.

FAM190A has alternative transcript forms. Transcript variants can physiologically be employed to create tissue regulatory specificity or protein diversity. In particular, the presence of 5′ alternative structures can derive from use of alternative promoters and/or from alternative splicing. The novel 5′ variants in cancer cells described herein may represent a loss of splicing fidelity (21), may subserve a tumorigenic role, or may be shared with certain normal cells.

Finally, it should be noted that some of the fusion joints match the minimum consensus peptide motives presented by the MHC (Table 1). The restricted set of fusion joints likely represents neo-antigens that could be clinically typed by diagnostic antibody panels, targeted by rearrangement specific therapies, or non-invasively monitored using personalized assays for disease burden (22).

TABLE 3
cDNAs primer sequences used in the PCR-based
analysis

| | | Chromosomal | |
|---|---|---|---|
| Primer | | position | |
| Name | Sequence | (Start bp)* | Target |
| FAM190A- | ATCCGCCTCTCCTCTCTCTC | +91048709 | 5′UTR |
| 01F | (SEQ ID NO: 96) | | |
| FAM190A- | CCCTTCACTTTTGCCAGAAC | −91229893 | Exon 2 |
| 01R | (SEQ ID NO: 97) | | |
| FAM190A- | TGAAGGGGATGATTCTGGTT | +91229906 | Exon 2 |
| 02F | (SEQ ID NO: 98) | | |
| FAM190A- | CCAAGTTTCCTTGGGGAGTT | −91230600 | Exon 2 |
| 02R | (SEQ ID NO: 99) | | |
| FAM190A- | CCCCAAGGAAACTTGGATTT | +91230604 | Exon 2 |
| 03F | (SEQ ID NO: 100) | | |
| FAM190A- | GAAAGGCACTGGCTCTTCAG | −91645120 | Exon 7 |
| 03R | (SEQ ID NO: 101) | | |
| FAM190A- | GATGAATGCTCGATGCTCAA | +91736925 | Exon 8 |
| 04F | (SEQ ID NO: 102) | | |
| FAM190A- | TCCTGCTGGCCTAGAGTTTG | −92520182 | Exon 11 |
| 04R | (SEQ ID NO: 103) | | |
| FAM190A- | AGCAATCTGTTCACTGTTAATGG | +92520556 | 3′UTR |
| 05F | (SEQ ID NO: 104) | | |
| FAM190A- | TTTGCCATCAAATGGGTTTT | −92521322 | 3′UTR |
| 05R | (SEQ ID NO: 105) | | |
| FAM190A- | TTTGACAAATAAGGCCCAAA | +92521350 | 3′UTR |
| 06F | (SEQ ID NO: 106) | | |
| FAM190A- | TAAATATTGCCCAGCCCAAA | −92522087 | 3′UTR |
| 06R | (SEQ ID NO: 107) | | |
| FAM190A- | CAGGCCTGGAGAAGGTGTAA | +92522584 | 3′UTR |
| 07F | (SEQ ID NO: 108) | | |
| FAM190A- | GGGGCAACTATGCCTACAAA | −92523331 | 3′UTR |
| 07R | (SEQ ID NO: 109) | | |
| FAM190A- | TTTGCGTGTTTGTAATTCTGC | +92521964 | 3′UTR |
| 08F | (SEQ ID NO: 110) | | |
| FAM190A- | CACTGAAAGAATGCCCACAT | −92522693 | 3′UTR |
| 08R | (SEQ ID NO: 111) | | |
| FAM190A- | CCTTTACAGGCAGGTTTGGA | +92520078 | Exon 11 |
| 09F | (SEQ ID NO: 112) | | |
| FAM190A- | CAAGCAAGCCACATTCTTCA | −92520705 | 3′UTR |
| 09R | (SEQ ID NO: 113) | | |
| FAM190A- | TGAAGAATGTGGCTTGCTTG | +92520705 | 3′UTR |
| 10F | (SEQ ID NO: 114) | | |
| FAM190A- | TTGAGTGCTCTTTTTCCCTTTC | −91229841 | Exon 2 |
| 11R | (SEQ ID NO: 115) | | |
| FAM190A- | AGGGGAAGACATTCTGTTGG | +91229769 | Exon 2 |
| 12F | (SEQ ID NO: 116) | | |
| FAM190A- | GAGTTTGTCCCCAGCATTGT | −91230585 | Exon 2 |
| 12R | (SEQ ID NO: 117) | | |
| FAM190A- | GAAGGCACTGCAGGGAGTAG | +91234082 | Exon 3 |
| 13F | (SEQ ID NO: 118) | | |
| FAM190A- | GCAGACATGAGTCCAGCAAG | +91645068 | Exon 7 |
| 14F | (SEQ ID NO: 119) | | |
| FAM190A- | GTCAGCCGTAAGCTGAAACC | +91049722 | 5′UTR* |
| 15F | (SEQ ID NO: 120) | | * |
| FAM190A- | GGGAGACCAGGGTAGATCGT | −91229456 | Exon 2 |
| 15R | (SEQ ID NO: 121) | | |
| FAM190A- | ACAATGCTGGGGACAAACTC | +91230585 | Exon 2 |
| 16F | (SEQ ID NO: 122) | | |
| FAM190A- | TACTCCCTGCAGTGCCTTCT | −91234081 | Exon 3 |
| 16R | (SEQ ID NO: 123) | | |
| FAM190A- | CCCAACTTCAGGAAGAGCTG | +91736977 | Exon 8 |
| 17F | (SEQ ID NO: 124) | | |
| FAM190A- | TTTTGTAGGGGCTTGTCTGG | −92519824 | Exon 11 |
| 17R | (SEQ ID NO: 125) | | |
| FAM190A- | AATCTGTCAGCGTGCACAAG | +92521643 | 3′UTR |
| 18F | (SEQ ID NO: 126) | | |
| FAM190A- | GCACATGTGTGTTCCTTTGG | −92521911 | 3′UTR |
| 18R | (SEQ ID NO: 127) | | |
| FAM190A- | CATCTAAAGTTCCCACCATAC | +92522033 | 3′UTR |
| 19F | (SEQ ID NO: 128) | | |
| FAM190A- | ACAGACACCAACATTTAGGAC | −92522165 | 3′UTR |
| 19R | (SEQ ID NO: 129) | | |
| FAM190A- | GAATGCCCAACAGTCCATCT | +91549238 | Exon 6 |
| 20F | (SEQ ID NO: 130) | | |
| FAM190A- | CGTCTGAAGTGAGGGTTTCA | −92519935 | Exon 11 |
| 21R | (SEQ ID NO: 131) | | |
| FAM190A- | GAGTCCAGCAAGCAGTACCA | +91645076 | Exon 7 |
| 22F | (SEQ ID NO: 132) | | |
| FAM190A- | AGAACGTGGAGAGCCCTTCT | −92520013 | Exon 11 |
| 23R | (SEQ ID NO: 133) | | |
| FAM190A- | GGCCTCTACAAGGTGTGGAA | +91549265 | Exon 6 |
| 24F | (SEQ ID NO: 134) | | |
| FAM190A- | CCGTAGCAACTTGGTCCTTT | −92520043 | Exon 11 |
| 24R | (SEQ ID NO: 135) | | |
| FAM190A- | GTGGCCCGTAAGTTCTGTGT | −92519957 | Exon 11 |
| 25R | (SEQ ID NO: 136) | | |

*Chromosomal position refers to GRCh37/hg19
**Refers to isoforms 3 and 4
When PCR product was sequenced, same primers used to amplify the amplicon were used.

TABLE 4
Human cell lines used for the analysis of FAM190A transcripts.

| Cancer | Tissue of | | | |
|---|---|---|---|---|
| cell line | Origin | Disease | Type of Transcript | Selected/Unselected |
| AGS | Stomach | Gastric Adenocarcinoma | Deletion exon 10 | Selected |
| AsPc1 | Pancreas | Adenocarcinoma | WT* | Unselected |
| BC-1 | Blood, B | Lymphoma | Deletion exon 10 | Selected |
| | lymphocyte | | | |
| BT-20 | Breast | Carcinoma | Deletion exon 6 | Unselected |
| BT-474 | Breast | Ductal carcinoma | WT | Unselected |
| BxPc3 | Pancreas | Adenocarcinoma | Deletion exons 9-10 | Selected |
| CAPAN1 | Pancreas | Adenocarcinoma | WT | Unselected |
| CAPAN2 | Pancreas | Adenocarcinoma | WT | Unselected |
| CFPAC1 | Pancreas | Ductal adenocarcinoma/ | WT | Unselected |
| | | cystic fibrosis | | |
| COLO205 | Colon | Colorectal adenocarcinoma | Deletion exons 8-9-10 | Selected |
| COLO357 | | | Deletion exons 8-9-10 | Unselected |
| DLD-1 | Colon | Colorectal adenocarcinoma | WT | Unselected |
| H508 | Cecum | Colorectal adenocarcinoma | Deletion exons 7-8-9- | Selected |
| | | | 10 | |
| H727 | Lung | Carcinoid | WT | Selected |
| H1581 | Lung | Non-small cell lung cancer | WT | Selected |
| H1975 | Lung | Adenocarcinoma/Non-small | Deletion exon 8, exons | Selected |
| | | cell lung cancer | 8-9, exons 8-9-10 | |
| H2126 | Lung | Adenocarcinoma/Non-small | Deletion exons 9-10 | Selected |
| | | cell lung cancer | | |
| H2228 | Lung | Adenocarcinoma/Non-small | Deletion exons 8-9 | Selected |
| | | cell lung cancer | | |
| HEK 293 | Embryonic | | Deletion exon 7-8 | Unselected |
| | kidney | | | |
| HeLa | Cervix | Adenocarcinoma | WT | Unselected |
| Hs 578T | Breast | Carcinoma | Undetected | Unselected |
| HT-1376 | Urinary | Carcinoma | Undetected | Selected |
| | bladder | | | |
| KATO III | Stomach | Gastric carcinoma | WT, deletion exons 9- | Selected |
| | | | 10 | |
| LNCA- | Prostate | Adenocarcinoma | Undetected | Selected |
| CLONE- | | | | |
| FGC | | | | |
| LoVo | Colon | Colorectal adenocarcinoma | Undetected | Selected |
| MCF7 | Breast | Adenocarcinoma | WT | Unselected |
| MDA- | Breast | Ductal carcinoma | WT | Unselected |
| MB-134- | | | | |
| VI | | | | |
| MDA- | Breast | Metastatic carcinoma | WT | Unselected |
| MB-453 | | | | |
| MiaPaCa2 | Pancreas | Carcinoma | Undetected | Unselected |
| Panc-1 | Pancreas | Epithelioid carcinoma | Deletion exons 4-5 | Unselected |
| P215 | Pancreas | Ductal carcinoma | WT | Unselected |
| PL45 | Pancreas | Ductal carcinoma | WT | Unselected |
| RKO | Colon | Carcinoma | Deletion exon 6 | Unselected |
| SW1417 | Colon | Colorectal adenocarcinoma | Deletion exon 9 | Selected |
| SW403 | Colon | Colorectal adenocarcinoma | Deletion exons 7-8-9 | Selected |
| SW620 | Colon | Colorectal adenocarcinoma | Undetected | Selected |
| SW780 | Urinary | Transitional cell carcinoma | WT, deletion exons 7,9, | Selected |
| | bladder | | deletion exon 9 | |
| SW837 | Rectum | Adenocarcinoma | Deletion exons 7-8-9- | Selected |
| | | | 10 | |
| T47D | Breast | Ductal carcinoma | WT, deletion exon 9 | Unselected |

*WT: wild-type

TABLE 5
gDNAs primer sequences used in the PCR-based analysis

| | | Chromosomal | |
|---|---|---|---|
| | | Position | |
| Primer Name | Sequence | (Start bp)* | Target |
| gFAM190A-01F | gggcatttacccagacagaa | +91228982 | Intron 1 |
| | (SEQ ID NO: 137) | | |
| gFAM190A-01R | TTTGCAGCctgagaaaggtt | −91229383 | Exon 2/ |
| | (SEQ ID NO: 138) | | Intron 2 |
| gFAM190A-02F | ACAATGCTGGGGACAAACTC | +91230585 | Exon 2 |
| | (SEQ ID NO: 139) | | |
| gFAM190A-02R | aaaatcaagcaggggaaaca | −91230907 | Intron 2 |
| | (SEQ ID NO: 140) | | |
| gFAM190A-03F | ATCCTGCCAAAGgtatgctg | +91230748 | Exon 2/ |
| | (SEQ ID NO: 141) | | Intron 2 |
| gFAM190A-03R | tttcttctgaaggtgctccaa | −91231036 | Intron 2 |
| | (SEQ ID NO: 142) | | |
| gFAM190A-04F | gtgagcaacgaagcaacaaa | +91233797 | Intron 2 |
| | (SEQ ID NO: 143) | | |
| gFAM190A-04R | TACTCCCTGCAGTGCCTTCT | −91234081 | Exon 3 |
| | (SEQ ID NO: 144) | | |
| gFAM190A-05F | ATCCTCGTCAGAAGGCACTG | +91234072 | Exon 3 |
| | (SEQ ID NO: 145) | | |
| gFAM190A-05R | tggaaacaaattttcccttca | −91234306 | Intron 3 |
| | (SEQ ID NO: 146) | | |
| gFAM190A-06F | AAGCAAAGAGCAGGTTCTTCA | +91234157 | Exon 3 |
| | (SEQ ID NO: 147) | | |
| gFAM190A-06R | tccatggaagcaaggaagac | −91234408 | Inton 3 |
| | (SEQ ID NO: 148) | | |
| gFAM190A-07F | catttttgcttcacggcttc | +91321099 | Intron 3 |
| | (SEQ ID NO: 149) | | |
| gFAM190A-07R | gaaagctactgaccagggtagg | −91321316 | Intron 4 |
| | (SEQ ID NO: 150) | | |
| gFAM190A-08F | TTGGGATCTTGTGAACTGGA | +91321202 | Exon 4 |
| | (SEQ ID NO: 151) | | |
| gFAM190A-08R | cgcacccaaattatgtctcc | −91321428 | Intron 4 |
| | (SEQ ID NO: 152) | | |
| gFAM190A-09F | aatgacttgagggagcctga | +91321825 | Intron 4 |
| | (SEQ ID NO: 153) | | |
| gFAM190A-09R | gcagtaggtcccaacgttct | −91322063 | Intron 4 |
| | (SEQ ID NO: 154) | | |
| gFAM190A-10F | tgttttcctgcccattcttc | +91320536 | Intron 4 |
| | (SEQ ID NO: 155) | | |
| gFAM190A-10R | aaaggaatttgctcactgcaa | −91320784 | Intron 4 |
| | (SEQ ID NO: 156) | | |
| gFAM190A-11F | gaaaaattgcccaaggtcat | +91389191 | Intron 4 |
| | (SEQ ID NO: 157) | | |
| gFAM190A-11R | AACTACTGCGGCACATGAGA | −91389415 | Exon 5 |
| | (SEQ ID NO: 158) | | |
| gFAM190A-12F | aaggggaaaaaggagatgtga | +91389311 | Intron 4 |
| | (SEQ ID NO: 159) | | |
| gFAM190A-12R | ctcacTGTTTGGAAGGCTCA | −91389491 | Intron 4/ |
| | (SEQ ID NO: 160) | | Exon 5 |
| gFAM190A-13F | aggaaggagaatcccttgga | +91388736 | Intron 4 |
| | (SEQ ID NO: 161) | | |
| gFAM190A-13R | tttgcaacatgaccacacct | −91389055 | Intron 4 |
| | (SEQ ID NO: 162) | | |
| gFAM190A-14F | ggtgtggtcatgttgcaaag | +91389056 | Intron 4 |
| | (SEQ ID NO: 163) | | |
| gFAM190A-14R | acTGTTTGGAAGGCTCAGGA | −91389488 | Exon 5/ |
| | (SEQ ID NO: 164) | | Intron 5 |
| gFAM190A-15F | CGTTGATCAAGAAGCCAGGT | +91549203 | Exon 6 |
| | (SEQ ID NO: 165) | | |
| gFAM190A-16F | tgctctaagcatgctgcact | +91549124 | Intron5 |
| | (SEQ ID NO: 166) | | |
| gFAM190A-16R | gagcagggtctaaccacgtc | −91549441 | Intron 6 |
| | (SEQ ID NO: 167) | | |
| gFAM190A-17F | GAATGCCCAACAGTCCATCT | +91549238 | Exon 6 |
| | (SEQ ID NO: 168) | | |
| gFAM190A-18F | GTGCAGACATGAGTCCAGCA | +91645066 | Exon 7 |
| | (SEQ ID NO: 169) | | |
| gFAM190A-18R | atttgaaatcagacccttgaaga | −91645243 | Intron 7 |
| | (SEQ ID NO: 170) | | |
| gFAM190A-19F | tgcacttaaaatatacatggagagtga | +91644829 | Intron 6 |
| | (SEQ ID NO: 171) | | |
| gFAM190A-19R | GAAAGGCACTGGCTCTTCAG | −91645120 | Exon 7 |
| | (SEQ ID NO: 172) | | |
| gFAM190A-20F | ctcccactgtgtgaagctga | +91736827 | Intron 7 |
| | (SEQ ID NO: 173) | | |
| gFAM190A-20R | ggattgaaccactggaagga | −91737098 | Intron 8 |
| | (SEQ ID NO: 174) | | |
| gFAM190A-21F | tcaagagcaaagtaaaagggcta | +91736856 | Intron 7 |
| | (SEQ ID NO: 175) | | |
| gFAM190A-21R | aaccgaaatgtttaatcctggtt | −91737235 | Intron 8 |
| | (SEQ ID NO: 176) | | |
| gFAM190A-22F | GATGAATGCTCGATGCTCAA | +91736925 | Exon 8 |
| | (SEQ ID NO: 177) | | |
| gFAM190A-22R | acctgaaccacacacactgc | −91737499 | Intron 8 |
| | (SEQ ID NO: 178) | | |
| gFAM190A-23F | CCCAACTTCAGGAAGAGCTG | +91736977 | Exon 8 |
| | (SEQ ID NO: 179) | | |
| gFAM190A-23R | aatgtggcctaagccttcaa | −91737274 | Intron 8 |
| | (SEQ ID NO: 180) | | |
| gFAM190A-24F | aaggtgggacagttgtgacc | +91844327 | Intron 8 |
| | (SEQ ID NO: 181) | | |
| gFAM190A-24R | ccaggaggtatttggggatt | −91844738 | Intron 9 |
| | (SEQ ID NO: 182) | | |
| gFAM190A-25F | cattttgcagGGAAAAGTCC | +91844511 | Intron 8/ |
| | (SEQ ID NO: 183) | | Exon 9 |
| gFAM190A-25R | gtgcaccgctaaatcctgtt | −91844696 | Intron 9 |
| | (SEQ ID NO: 184) | | |
| gFAM190A-26F | TACTCAGGGCCATCGCTTAG | +92015880 | Intron 10 |
| | (SEQ ID NO: 185) | | |
| gFAM190A-26R | TAATTGGAGCAACGGGAGTC | −92016170 | Intron 10 |
| | (SEQ ID NO: 186) | | |
| gFAM190A-27F | gtactgcaagggcttctgct | +92006680 | Intron 9 |
| | (SEQ ID NO: 187) | | |
| gFAM190A-28F | gcgctgagatctgctggtag | +92006727 | Intron 9 |
| | (SEQ ID NO: 188) | | |
| gFAM190A-28R | tgctttggcagaatgaacag | −92007288 | Intron10 |
| | (SEQ ID NO: 189) | | |
| gFAM190A-29F | caggaaatgcattgggacta | +92006894 | Intron 9 |
| | (SEQ ID NO: 190) | | |
| gFAM190A-29R | ttttcccacccctcttcttt | −92007158 | Intron10 |
| | (SEQ ID NO: 191) | | |

*Chromosomal position refers to GRCh37/hg19
Small nd capital letters refer to intronic and exonic sequence, respectively.

TABLE 6
cDNA primer sequences used in the 5′RACE analysis

| | | Chromosomal | |
|---|---|---|---|
| | | Position | |
| Primer Name | Sequence | (Start) bp | Target |
| RACEFAM190A- | GGCTCTTGCTTAGGCTCACTCCCCTTC | −91229654 | Exon 2 |
| 01R | (SEQ ID NO: 192) | | |
| RACEFAM190A- | TATTGGCAACCGGGAGACCAGGGTAG | −91229461 | Exon 2 |
| 02R | (SEQ ID NO: 193) | | |
| FAM190A-01R | CCCTTCACTTTTGCCAGAAC | −91229893 | Exon 2 |
| | (SEQ ID NO: 194) | | |

TABLE 7
cDNA mouse primer sequences used in the RCR-based analysis

| | | Chromosomal | |
|---|---|---|---|
| | | Position | |
| Primer Name | Sequence | (Start) bp* | Target |
| mFam190a1F | CCAGTGGATCCAGGAAAAGA | +61373007 | Exon 5 |
| | (SEQ ID NO: 195) | | |
| mFam190a1R | TGGTTCGATTTCGGTGTGTA | −62329781 | Exon 11 |
| | (SEQ ID NO: 196) | | |
| mFam190a2F | CTTGCCAGTAGCCTCAGTCC | 61263650 | Exon 3 |
| | (SEQ ID NO: 197) | | |
| mFam190a2R | CAGAAGCAGCGTCTTCACTG | −61520828 | Exon 6 |
| | (SEQ ID NO: 198) | | |
| mFam190a3F | CGCTCAGAATGACTGACTGC | +61130464 | 5′ UTR |
| | (SEQ ID NO: 199) | | |
| mFam190a3R | CCTGGTTTTGACGTGTTCCT | −61261165 | Exon 2 |
| | (SEQ ID NO: 200) | | |
| mFam190a4F | GCAGGAGGAGTGCTGAAGAC | +61130525 | 5′ UTR |
| | (SEQ ID NO: 201) | | |
| mFam190a4R | TGTCTTCCCCTGGTTTTGAC | −61261173 | Exon 2 |
| | (SEQ ID NO: 202) | | |
| mFam190a5F | ACCACCTCACTTCCCATCAG | +61130525 | Exon 7 |
| | (SEQ ID NO: 203) | | |
| mFam190a5R | TGGGTCCTTTGGTTTTTCTG | −62330086 | Exon 11 |
| | (SEQ ID NO: 204) | | |
| mFam190a6R | GATGGGGAAGGTCTCCCTAA | −61261515 | Exon 2 |
| | (SEQ ID NO: 205) | | |
| mFam190a7F | TAGGGAGACCTTCCCCATCT | +61261516 | Exon 2 |
| | (SEQ ID NO: 206) | | |
| mFam190a7R | CCAGTTCACAGGATCCCAGA | 61323969 | Exon 4 |
| | (SEQ ID NO: 207) | | |
| mFam190a8F | AACGCTTTAAAGGGGTCCAC | 61262059 | Exon 1 |
| | (SEQ ID NO: 208) | | |
| mFam190a8R | TCTTGAAGCAAACGCCTTCT | 61760644 | Exon 8 |
| | (SEQ ID NO: 209) | | |
| mFam190a9F | TGCCATTCAGACTGATGCTC | 61520795 | Exon 5 |
| | (SEQ ID NO: 210) | | |
| mFam190a9R | GGGCATGACCACTCTTAGGT | 62330311 | 3′UTR |
| | (SEQ ID NO: 211) | | |
| mFam190a10F | AGAGTGATGTGAGCCCTTCC | 61588431 | Exon6/7 |
| | (SEQ ID NO: 212) | | |
| mFam190a10R | TCAGTCAACATACTCAAGCTGTCA | 62330395 | 3/UTR |
| | (SEQ ID NO: 213) | | |
| mFam190a11F | AGTGAAGACGCTGCTTCTGA | 61520829 | Exon 6 |
| | (SEQ ID NO: 214) | | |
| mFam190a11R | AAAAGCCCATAAGTCTTTGCT | 62330358 | 3′UTR |
| | (SEQ ID NO: 215) | | |
| mActb1F | CTGTATTCCCCTCCATCGTG | −143667261** | |
| | (SEQ ID NO: 216) | | |
| mActb1R | AAGGAAGGCTGGAAAAGAGC | +143666028** | |
| | (SEQ ID NO: 217) | | |

*Chromosomal position according to NCBI37/mm9
**Actb is on chromosome 5

### Example 5

### Intragenic Rearrangements Produce In-Frame Deletions with a Potential Oncogenic Function

The FAM190A gene sequence information was taken from UCSC Genome browser database (http://genome.ucsc.edu/). Genomic DNA from lung and pancreatic cancer cell lines and xenografts was purified. Total RNA was extracted from cell lines and xenografts using Trizol (Gibco BRL, Life Technologies, Gaithersburg, Md., USA) and chloroform. Purification was performed on RNAeasy columns according to manufacturer's instructions (Qiagen, Valencia, Calif.). RNA quality was assessed by electrophoresis on a 1% non-denaturing agarose gel stained with ethidium bromide. For cDNA production, 1 ug of total RNA was treated with DNase I for 30 min at 25C (Invitrogen, Carlsbad, Calif.). Then, 3 ul of digested RNA, DNase I treated, were retro-transcribed to produce cDNA using SuperScript® III First-Strand Synthesis System according to manufacturer's instructions (Invitrogen, Carlsbad, Calif.). The primers were generated using the Primer3 web-based primer picking service (http://frodo.wi.mit.edu/primer3/). Sequences of the wildtype FAM190A coding region cDNA were used in a yeast two-hybrid analysis. Interacting proteins were identified.

A rabbit monoclonal antibody against the C-terminal third of the FAM190A protein was raised and was used as the primary antibody in immunofluorescence. An anti-rabbit antibody conjugated to a fluorescent dye was used as the secondary antibody. DAPI nuclear stain was used to mark the DNA of the chromosomes. Images were captured using an inverted Zeiss fluorescence microscope and digital camera. Images of FAM190A immunopositivity and of DAPI signals were combined and were false-colored by computer software (MetaMorph).

A set of primers covering the entire coding sequence and spanning exon junctions were designed from cDNA sequence to amplify large fragments of the KIAA1680 gene using a PCR-based assay. 39 cell lines and 25 xenografts were studied, from multiple tumor types. The PCR products were analyzed by sequencing. This analysis revealed, along with the wild-type sequence, eight different rearranged in-frame structures: a transcript where exon 6 was joined to exon 9, a transcript where exon 6 was joined to exon 10, a transcript where exon 6 was joined to exon 11, a transcript where exon 7 was joined to exon 10, a transcript where exon 7 was joined to exon 11, a transcript where exon 8 was joined to exon 11, a transcript where exon 8 was joined to exon 10 and a transcript where exon 9 was joined to exon 11. Two different rearrangements causing frame-shift of the coding sequence were also observed which occurred outside exon 7-10 (Table 9) and an in-frame deletion in which exon 3 was joined to exon 8. The sequences of the primers used to characterize the rearrangements are listed (Table 8).

No mutation was found in the coding sequence of the remaining part of the gene. In some samples, rearranged and unrearranged transcripts were both found. In addition, 48 normal human tissue samples were surveyed finding that 35 were expressed. In 4 out of 35 samples, rearranged structures were observed which were unconfirmed in other samples of the same tissue and of a different kind if compared to the one observed in the cancer samples.

In the two-hybrid analysis, interacting proteins included the centrosomal protein Cep70 and the protein Ndel1, which interacts with the mitotic spindle in late prophase as the asters begin to form as organized by the centrosomes.

For immunofluorescence, a monoclonal antibody was chosen that had been raised against FAM190A and that identified a Flag-tagged FAM190A protein exogenously expressed in cultured mammalian cells. In immunofluorescent studies, the antibody identified signals including occasional cells having one to two cytoplasmic point locations (possibly representing centrosomes) and early mitotic asters in late prophase to metaphase.

TABLE 3
KIAA1680 Forward and Reverse Primers

| | | Strand/Chromosomal | |
|---|---|---|---|
| | | Position | Side ofthe |
| Primer name | Sequence | (hg19) | deletion |
| KIAA91955948F | GATGAATGCTCGATGCTCAA | +91736925 | 1 |
| | (SEQ ID NO: 67) | | |
| KIAA91955948R | TCCTGCTGGCCTAGAGTTTG | −92520182 | 2 |
| | (SEQ ID NO: 68) | | |
| KIAA92103462F | GCAGACATGAGTCCAGCAAG | +91645068 | 1 |
| | (SEQ ID NO: 69) | | |
| KIAA92103462R | CGTCTGAAGTGAGGGTTTCA | −92519935 | 2 |
| | (SEQ ID NO: 70) | | |
| KIAA92103470F | GAGTCCAGCAAGCAGTACCA | +91645076 | 1 |
| | (SEQ ID NO: 71) | | |
| KIAA92195319R | AGAACGTGGAGAGCCCTTCT | −92520013 | 2 |
| | (SEQ ID NO: 72) | | |
| KIAA91549265F | GGCCTCTACAAGGTGTGGAA | +91549265 | 1 |
| | (SEQ ID NO: 73) | | |
| KIAA91549265R | CCGTAGCAACTTGGTCCTTT | −92520043 | 2 |
| | (SEQ ID NO: 74) | | |
| KIAA91645068R | GTGGCCCGTAAGTTCTGTGT | −92519957 | 2 |
| | (SEQ ID NO: 75) | | |
| KIAA91736925R | TTTTGTAGGGGCTTGTCTGG | −92519824 | 2 |
| | (SEQ ID NO: 76) | | |
| KIAA_Left7 | GAATGCCCAACAGTCCATCT | +91549238 | 1 |
| | (SEQ ID NO: 77) | | |
| KIAA91549265F | GGCCTCTACAAGGTGTGGAA | +91549265 | 2 |
| | (SEQ ID NO: 78) | | |

Table 1 includes the sequences of the forward and reverse primers spanning the junctions of exons 6-11 and 8-11 of KIAA1680. There are two sides of the deletion. Side 1, is the left side (which proceeds from the beginning of the gene until the end of exon 7) and side 2 is the right side (which proceeds from the beginning of the exon 11 until the end of the gene). According to this terminology all primers marked with “F” or forward at the end, will anneal to side 1, and all primers marked with “R” or reverse at the end, anneal to side 2.

TABLE 2
Variations of FAM190A coding region transcript

| Coding | | | Exon | Joint |
|---|---|---|---|---|
| Variants | Samples Affected | Type | Affected | Created |
| A | 1 artificially | In-frame deletion | 7-8 | LQE GKV |
| | immortalized line | | | (SEQ ID NO: 79) |
| B | 1 cell line | In-frame deletion | 7-8-9 | LQE GLN |
| | | | | (SEQ ID NO: 80) |
| C | 2 cell lines | In-frame deletion | 7-8-9-10 | LQE ATY |
| | | | | (SEQ ID NO: 81) |
| D | 1 cell line | In-frame deletion | 8-9 | PFK GLN |
| | | | | (SEQ ID NO: 82) |
| E | 2 cell lines | In-frame deletion | 8-9-10 | PFK ATY |
| | | | | (SEQ ID NO: 83) |
| F | 3 cell lines and 4 | In-frame deletion | 9-10 | EEL ATY |
| | xenografted tumor | | | (SEQ ID NO: 84) |
| G | 2 cell lines and 10 | In-frame deletion | 9 | EEL GLN |
| | xenografted tumors | | | (SEQ ID NO: 85) |
| H | 2 cell lines | In-frame deletion | 10 | CYD ATY |
| | | | | (SEQ ID NO: 86) |
| I | 1 cell line | In-frame deletion | 4-7 | NSL DIM |
| | | | | (SEQ ID NO: 87) |
| J | Not seen in cancer | In-frame deletion | 4-6 | NSL SAD |
| | | | | (SEQ ID NO: 88) |
| K | Not seen in cancer | In-frame deletion | 4-8 | NSL GKV |
| | | | | (SEQ ID NO: 89) |
| L | Not seen in cancer | In-frame deletion | 4-9 | NSL GLN |
| | | | | (SEQ ID NO: 90) |
| M | Not seen in cancer | In-frame deletion | 4-10 | NSL ATY |
| | | | | (SEQ ID NO: 91) |
| N | Not seen in cancer | In-frame deletion | 7 | LQE DIM |
| | | | | (SEQ ID NO: 92) |
| O | Not seen in cancer | In-frame deletion | 8 | PFK GKV |
| | | | | (SEQ ID NO: 93) |
| P | 1 cell line | Out of frame | 4-5 | NSL ESF |
| | | deletion | | (SEQ ID NO: 94) |
| Q | 1 cell line | Out of frame | 6 | SK QVQ |
| | | deletion | | (SEQ ID NO: 95) |

Single underline indicates the last three amino acidic residues of the exon that precedes the deletion.
Double underline indicates the first three amino acidic residues of the exon that follow the deletion.
Stagger Line indicates the first three amino acidic residues that would be created due to the frame-shift

### REFERENCES

1. Glover T W. Common fragile sites. Cancer Lett. 2006; 232: 4-12.
2. Kannan K, Munirajan A K, Bhuvarahamurthy V, Mohanprasad B K, Shankar P, Tsuchida N, Shanmugam G. FHIT gene mutations and single nucleotide polymorphism in Indian oral and cervical squamous cell carcinomas. Oral Oncol. 2000; 36: 189-193.
3. Kannan K, Krishnamurthy J, Feng J, Nakajima T, Tsuchida N, Shanmugam G. Mutation profile of the p53, fhit, p161NK4a/p19ARF and H-ras genes in Indian breast carcinomas. Int J. Oncol. 2000; 17: 1031-1035.
4. Iliopoulos D, Guler G, Han S Y, Druck T, Ottey M, McCorkell K A, Huebner K. Roles of FHIT and WWOX fragile genes in cancer. Cancer Lett. 2006; 232: 27-36.
5. Richards R I. Fragile and unstable chromosomes in cancer: causes and consequences. Trends Genet. 2001; 17: 339-345.
6. Rozier L, El-Achkar E, Apiou F, Debatisse M. Characterization of a conserved aphidicolin-sensitive common fragile site at human 4q22 and mouse 6C1: possible association with an inherited disease and cancer. Oncogene. 2004; 23: 6872-6880.
7. Bignell G R, Greenman C D, Davies H, Butler A P, Edkins S, Andrews J M, Buck G, Chen L, Beare D, Latimer C, Widaa S, Hinton J, Fahey C, Fu B, Swamy S, Dalgliesh G L, et al. Signatures of mutation and selection in the cancer genome. Nature. 2010; 463: 893-898.
8. Nancarrow D J, Handoko H Y, Smithers B M, Gotley D C, Drew P A, Watson D I, Clouston A D, Hayward N K, Whiteman D C. Genome-wide copy number analysis in esophageal adenocarcinoma using high-density singlenucleotide polymorphism arrays. Cancer Res. 2008; 68: 4163-4172.
9. Calhoun E S, Hucl T, Gallmeier E, West K M, Arking D E, Maitra A, Iacobuzio-Donahue C A, Chakravarti A, Hruban R H, Kern S E. Identifying allelic loss and homozygous deletions in pancreatic cancer without matched normals using high-density single-nucleotide polymorphism arrays. Cancer Res. 2006; 66: 7920-7928.
10. Beroukhim R, Mermel C H, Porter D, Wei G, Raychaudhuri S, Donovan J, Barretina J, Boehm J S, Dobson J, Urashima M, Mc Henry K T, Pinchback R, Ligon A H, Cho Y J, Haery L, Greulich H, et al. The landscape of somatic copy-number alteration across human cancers. Nature. 2010; 463: 899-905.
11. Wong A J, Ruppert J M, Bigner S H, Grzeschik C H, Humphrey P A, Bigner D S, Vogelstein B. Structural alterations of the epidermal growth factor receptor gene in human gliomas. Proc Natl Acad Sci USA. 1992; 89: 2965-2969.
12 Zhao X, Weir B A, LaFramboise T, Lin M, Beroukhim R, Garraway L, Beheshti J, Lee J C, Naoki K, Richards W G, Sugarbaker D, Chen F, Rubin M A, Janne P A, Girard L, Minna J, et al. Homozygous deletions and chromosome amplifications in human lung carcinomas revealed by single nucleotide polymorphism array analysis. Cancer Res. 2005; 65: 5561-5570.
13. Campbell P J, Yachida S, Mudie L J, Stephens P J, Pleasance E D, Stebbings L A, Morsberger L A, Latimer C, McLaren S, Lin M L, McBride D J, Varela I, Nik-Zainal S A, Leroy C, Jia M, Menzies A, et al. The patterns and dynamics of genomic instability in metastatic pancreatic cancer. Nature. 2010; 467:1109-13.
14. Driouch K, Prydz H, Monese R, Johansen H, Lidereau R, Frengen E. Alternative transcripts of the candidate tumor suppressor gene, WWOX, are expressed at high levels in human breast tumors. Oncogene. 2002; 21: 1832-1840.
15. Sozzi G, Huebner K, Croce C M. FHIT in human cancer. Adv Cancer Res. 1998; 74: 141-166.
16. Wang L, Darling J, Zhang J S, Qian C P, Hartmann L, Conover C, Jenkins R, Smith D I. Frequent homozygous deletions in the FRA3B region in tumor cell lines still leave the FHIT exons intact. Oncogene. 1998; 16: 635-642.
17. Hilgers W, Kern S E. Molecular genetic basis of pancreatic adenocarcinoma. Genes Chromosomes Cancer. 1999; 26: 1-12.
18. Le Beau M M, Drabkin H, Glover T W, Gemmill R, Rassool F V, McKeithan T W, Smith D I. An FHIT tumor suppressor gene? Genes Chromosomes Cancer. 1998; 21: 281-289.
19. Jones S, Hruban R H, Kamiyama M, Borges M, Zhang X, Parsons D W, Lin J C, Palmisano E, Brune K, Jaffee E M, Iacobuzio-Donahue C A, Maitra A, Parmigiani G, Kern S E, Velculescu V E, Kinzler K W, et al. Exomic sequencing identifies PALB2 as a pancreatic cancer susceptibility gene. Science. 2009; 324: 217.
20. Uckun F M, Goodman P, Ma H, Dibirdik I, Qazi S CD22 EXON 12 deletion as a pathogenic mechanism of human B-precursor leukemia. Proc Natl Acad Sci U S A. 2010; 107: 16852-16857.
21. Lee M P, Feinberg A P. Aberrant splicing but not mutations of TSG101 in human breast cancer. Cancer Res. 1997; 57: 3131-3134.
22. Leary R J, Kinde I, Diehl F, Schmidt K, Clouser C, Duncan C, Antipova A, Lee C, McKernan K, De La Vega F M, Kinzler K W, Vogelstein B, Diaz L A Jr, Velculescu V E. Development of personalized tumor biomarkers using massively parallel sequencing. Sci Transl Med. 2010; 2: 20ra14.
23. Caldas C, Hahn S A, da Costa L T, Redston M S, Schutte M, Seymour A B, Weinstein C L, Hruban R H, Yeo C J, Kern S E. Frequent somatic mutations and homozygous deletions of the p16 (MTS1) gene in pancreatic adenocarcinoma. Nat. Genet. 1994; 8: 27-32.
24. Iacobuzio-Donahue C A, van der Heijden M S, Baumgartner M R, Troup W J, Romm J M, Doheny K, Pugh E, Yeo C J, Goggins M G, Hruban R H, Kern S E. Large-scale allelotype of pancreaticobiliary carcinoma provides quantitative estimates of genome-wide allelic loss. Cancer Res. 2004; 64: 871-875.
25. Brody J R, Calhoun E S, Gallmeier E, Creavalle T D, Kern S E. Ultra-fast high-resolution agarose electrophoresis of DNA and RNA using low-molarity conductive media. Biotechniques. 2004; 37: 598, 600, 602.
26. Rammensee H G, Friede T, Stevanoviic S. MHC ligands and peptide motifs: first listing. Immunogenetics. 1995; 41: 178-228.

We claim:

1. An isolated nucleic acid molecule comprising a sequence selected from the group consisting of: SEQ ID NOS:2-16.

2. An isolated amino acid molecule comprising a sequence selected from the group consisting of SEQ ID NOS:18-27.

3. An isolated nucleic acid molecule that encodes an amino acid sequence selected from the group consisting of: SEQ ID NOS:18-27.

4. An isolated nucleic acid molecule comprising a sequence selected from the group consisting of: SEQ ID NOS:29-38.

5. An isolated amino acid molecule comprising a sequence selected from the group consisting of: SEQ ID NOS:39-48.

6. An isolated nucleic acid molecule that encodes an amino acid sequence selected from the group consisting of SEQ ID NOS:39-48.

7. An expression vector comprising a nucleic acid molecule selected from the group consisting of SEQ ID NOS:2-16.

8. A host cell transformed with the expression vector of claim 7.

9. An expression vector comprising a nucleic acid molecule selected from the group consisting of SEQ ID NOS:29-38.

10. A host cell transformed with the expression vector of claim 9.

11. A host cell comprising a cDNA which encodes an immunogenic agent, wherein the host cell expresses the immunogenic agent, wherein the immunogenic agent is selected from the group consisting of SEQ ID NOS: 18-27.

12. A host cell comprising a cDNA which encodes an immunogenic agent, wherein the host cell expresses the immunogenic agent, wherein the immunogenic agent is selected from the group consisting of SEQ ID NOS: 39-48.

13. A method of generating antibodies comprising the steps of (a) culturing a host cell comprising a cDNA encoding an immunogenic agent whereby the immunogenic agent is expressed in the host cell, and whereby the immunogenic agent is selected from the group consisting of SEQ ID NOS: 18-27; and (b) administering the immunogenic agent to a non-human animal, wherein the animal produces antibodies which specifically bind to the immunogenic agent.

14. The method of claim 13, wherein the immunogenic agent is administered in a purified protein preparation.

15. The method of claim 13, further comprising (c) collecting B cells from the animal; (d) fusing the B cells with myeloma cells to make hybridomas; and (e) collecting the antibodies from the hybridomas.

16. The method of claim 13, wherein the non-human animal is a mouse.

17. An antibody composition made from the process of claim 13.

18. A method for diagnosing cancer or a likelihood thereof in a patient comprising the steps of (a) obtaining a biological sample from the patient; (b) subjecting the sample to an assay for detecting any of the FMA190A mutant proteins selected from the group consisting of SEQ ID NOS: 18-25; and (d) determining that the patient has cancer or a likelihood thereof if a FMA190A mutant protein is detected.

19. A method for diagnosing cancer or a likelihood thereof in a patient comprising the steps of (a) obtaining a biological sample from the patient; (b) subjecting the sample to an assay for detecting any of the FMA190A mutant nucleic acid sequences selected from the group consisting of SEQ ID NOS: 2-10; and (d) determining that the patient has cancer or a likelihood thereof if a FMA190A mutant nucleic acid sequence is detected.

20. A method for identifying an agent useful for treating cancer in a patient comprising the steps of (a) providing a cell which expresses FAM190A protein selected from the group consisting of SEQ ID NOS:18-27; (b) exposing the cell to candidate agents; and (c) identifying an agent that inhibits a biological function or reduces the level or expression of the FAM190A protein.

21. A method for treating a patient having a cancer characterized by a FAM190A intragenic rearrangement comprising the step of administering to the patient an agent that inhibits a biological function or reduces the level or expression of the FAM190A protein.

22. The method of claim 21, wherein the agent is selected from the group consisting of an antibody, an inhibitory nucleic acid molecule, and a small molecule.

23. A transgenic mouse that expresses a mutant FAM190A having an amino acid sequence selected from the group consisting of SEQ ID NOS: 18-27.

24. An isolated antibody, or biologically active fragment thereof, which specifically binds a FAM190A mutant protein having an amino acid sequence selected from the group consisting of SEQ ID NOS:18-27.

25. The antibody of claim 24, wherein the antibody is a monoclonal antibody.

26. An inhibitory nucleic acid molecule that is complementary to at least a fragment of a FAM190A nucleic acid molecule selected from the group consisting of SEQ ID NOS:2-16, and that decreases FAM190A expression in a cell.

27. The inhibitory nucleic acid molecule of claim 26, wherein the nucleic acid molecule is an siRNA.

28. The inhibitory nucleic acid molecule of claim 26, wherein the nucleic acid molecule is an shRNA.


SEQUENCE LISTING (259 sequences)

SEQ ID NO: 1 - DNA, 2703 bp, Homo sapiens
  misc_feature (1)..(2703): Human FAM190A wild type
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaaggatataatgaaagatgaatgctcgatgctcaagctgcagctgaaagagaaggatgaactcatttcccaacttcaggaagagctgggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 2 - DNA, 2541 bp, Homo sapiens
  misc_feature (1)..(2541): An in-frame intragenic rearrangement of the human FAM190A gene. This deletion removes exon 7 and 8, resulting in a mRNA where exon 6 is spliced to exon 9. This mutant transcript lacks 162bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 3 - DNA, 2463 bp, Homo sapiens
  misc_feature (1)..(2463): An in-frame intragenic rearrangement of the human FAM190A gene. This deletion removes exon 7, 8 and 9 resulting in a mRNA where exon 6 is spliced to exon 10. This mutant transcript lacks 240bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 4 - DNA, 2418 bp, Homo sapiens
  misc_feature (1)..(2418): An in-frame intragenic rearrangement of the human FAM190A gene. This deletion removes exon 7, 8, 9 and 10 resulting in a mRNA where exon 6 is spliced to exon 11. This mutant transcript lacks 285bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 5 - DNA, 2541 bp, Homo sapiens
  misc_feature (1)..(2541): An in-frame intragenic rearrangement of the human FAM190A gene. This deletion removes exon 8 and 9 resulting in a mRNA where exon 7 is spliced to exon 10. This mutant transcript lacks 162bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaagggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 6 - DNA, 2496 bp, Homo sapiens
  misc_feature (1)..(2496): An in-frame intragenic rearrangement of the human FAM190A gene. This deletion removes exon 8, 9 and 10 resulting in a mRNA where exon 7 is spliced to exon 11. This mutant transcript lacks 207bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 7 - DNA, 2580 bp, Homo sapiens
  misc_feature (1)..(2580): An in-frame intragenic rearrangement of the human FAM190A gene. This deletion removes exon 9 and 10 resulting in a mRNA where exon 8 is spliced to exon 11. This mutant transcript lacks 123bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaaggatataatgaaagatgaatgctcgatgctcaagctgcagctgaaagagaaggatgaactcatttcccaacttcaggaagagctggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 8 - DNA, 2625 bp, Homo sapiens
  misc_feature (1)..(2625): An in-frame intragenic rearrangement of the human FAM190A gene. This deletion removes only exon 9 resulting in a mRNA where exon 8 is spliced to exon 10. This mutant transcript lacks 78bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaaggatataatgaaagatgaatgctcgatgctcaagctgcagctgaaagagaaggatgaactcatttcccaacttcaggaagagctgggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 9 - DNA, 3144 bp, Homo sapiens
  misc_feature (1)..(3144): An in-frame intragenic rearrangement of the human FAM190A gene. This deletion removes only exon 10 resulting in a mRNA where exon 9 is spliced to exon 11. This mutant transcript lacks 45bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaaggatataatgaaagatgaatgctcgatgctcaagctgcagctgaaagagaaggatgaactcatttcccaacttcaggaagagctgggaaaagtccggcatttacagaaggcttttgcttcaagagtagataaatccacacagactgaactactatgctatgatgctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaagctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 10 - DNA, 2202 bp, Homo sapiens
  misc_feature (1)..(2202): An in-frame intragenic rearrangement of the human FAM190A gene. This deletion removes exons 4 through 7 resulting in a mRNA where exon 3 is spliced to exon 8. This mutant transcript lacks 501bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatataatgaaagatgaatgctcgatgctcaagctgcagctgaaagagaaggatgaactcatttcccaacttcaggaagagctgggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 11 - DNA, 2280 bp, Homo sapiens
  misc_feature (1)..(2280): An in-frame intragenic rearrangement of the human FAM190A gene. This deletion removes exons 4 through 6 resulting in a mRNA where exon 3 is spliced to exon 7. This mutant transcript lacks 423bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttgagtgcagacatgagtccagcaagcagtaccacgtcacttcctgttagtcctcttactgaagagccagtgcctttcaaggatataatgaaagatgaatgctcgatgctcaagctgcagctgaaagagaaggatgaactcatttcccaacttcaggaagagctgggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 12 - DNA, 2118 bp, Homo sapiens
  misc_feature (1)..(2118): An in-frame intragenic rearrangement of the human FAM190A gene. This deletion removes exons 4 through 8 resulting in a mRNA where exon 3 is spliced to exon 9. This mutant transcript lacks 585bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttgggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 13 - DNA, 2040 bp, Homo sapiens
  misc_feature (1)..(2040): An in-frame intragenic rearrangement of the human FAM190A gene. This deletion removes exons 4 through 9 resulting in a mRNA where exon 3 is spliced to exon 10. This mutant transcript lacks 663bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttgggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 14 - DNA, 1995 bp, Homo sapiens
  misc_feature (1)..(1995): An in-frame intragenic rearrangement of the human FAM190A gene. This deletion removes exons 4 through 10 resulting in a mRNA where exon 3 is spliced to exon 11. This mutant transcript lacks 708bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 15 - DNA, 2625 bp, Homo sapiens
  misc_feature (1)..(2625): An in-frame intragenic rearrangement of the human FAM190A gene. This deletion removes only exon 7 resulting in a mRNA where exon 6 is spliced to exon 8. This mutant transcript lacks 78bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagaggatataatgaaagatgaatgctcgatgctcaagctgcagctgaaagagaaggatgaactcatttcccaacttcaggaagagctgggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 16 - DNA, 2619 bp, Homo sapiens
  misc_feature (1)..(2619): An in-frame intragenic rearrangement of the human FAM190A gene. This deletion removes only exon 8 resulting in a mRNA where exon 7 is spliced to exon 9. This mutant transcript lacks 84bp.
atgggggactcaggatcaagacgatctaccctggtctcccggttgccaatattcagaagaagtattaacagaagacatgattctcttccttcttcaccttcttccagtaatacagttggtgtccacagttcctctccttccagcactaactcaagctcaggtagcacaggtaaacggaggagcatattccgtactccttccattagcttccaccataagaaggggagtgagcctaagcaagagcctaccaaccagaaccttagtatttcaaatggtgctcaacctggtcacagcaatatgcagaaactgagtttggaagaacatattaagaccaggggaagacattctgttggttttagtagttcacgaaataagaagataacaagatctttgacagaggattttgaaagggaaaaagagcactcaactaacaagaatgtctttataaattgtctaagttctggcaaaagtgaaggggatgattctggtttcacagaagaccaaactcgtcgttctgttaagcagtcaacaaggaagctactccctaaatctttttcatctcactataaattttctaagccagttctacagagccaatccatttcattggtacaacagtctgaattctcattggaagttacacagtaccaagagagagaacctgtattagtaagagcttcgccatcctgttctgtggatgtaacagaacgggcaggaagctctttacaatctcctttgctttctgctgatcttaccacagctcagacaccttcagaatttttagccttgactgaagattctgtgtctgaaatggatgcattttctaaaagtggaagcatggcatcccactgtgacaactttggccacaatgattctacctctcagatgtccctcaattctgctgctgttacaaagacaacaacagaacttacgggaactgttccctgtgcaattatgtctcctgggaaatataggttagagggtcaatgtagcactgaatctaattcattaccggaaacctctgctgctaatcagaaggaagtgttattacaaattgctgaactacctgctacaagtgtgagccactcagagagtaacctaccagcagatagtgaaagagaagaaaatatagggttacaaaatggtgaaacaatgctggggacaaactccccaaggaaacttggattttatgagcaacataaagcaatagcggaacatgtaaaagggatccatcctatttcagattcaaagataatacctacttctggtgatcatcatatttttaacaaaacatcacatggatatgaagcaaatcctgccaaagttcttgccagtagtctcagtccatttcgtgaaggaagatttatagagaggagactgcgatcctcgtcagaaggcactgcagggagtagcagaatgattttgaaaccgaaagatggaaatatagaagaagttaatagtttaagaaagcaaagagcaggttcttcatcttcaaaaatgaacagtttggatgttttgaataatttgggatcttgtgaactggatgaagatgatctaatgcttgatcttgaatttttagaggaacagagtcttcacccttctgtttgccgggaggactcatatcactctgtcgtctcatgtgccgcagtagttcttactcctatggaaccaatgatagaaatgaagaaaagagaagaaccagaatttcctgagccttccaaacagaatctttccctgaaattaacaaaggacgttgatcaagaagccaggtgttcccacatcagccgaatgcccaacagtccatctgcggattggcctctacaaggtgtggaagaaaacggaggcatagattctctgccattcagactgatgttacaggactgcacggcagtcaagacgttattattaaagatgaagagagttcttcaagagagtgcagacatgagtccagcaagcagtaccacgtcacttcctgttagtcctcttactgaagagccagtgcctttcaagggaaaagtccggcatttacagaaggcttttgcttcaagagtagataaatccacacagactgaactactatgctatgatggtttaaacttgaaaagactagagacagtacaaggagggagagaggctacatatcgaaatcgaattgtgagccaaaatctcagcacaagggacagaaaagcaatacatactcccaccgaggaccgttttaggtattcggcagcggaccagacaagcccctacaaaaacaagacctgtcaactcccaagtctctgtttaagtaatttcctgaaggacaaggaactagcagaagttatcaaacattcaagaggaacttatgaaaccctcacttcagacgttacacagaacttacgggccaccgttgggcagagctctctgaagccaacagctaagacagaagggctctccacgttcttagagaaaccaaaggaccaagttgctacggcccgacagcattcgacctttacaggcaggtttggacagccacccagagggccaatctctttacacatgtacagcaggaagaatgtgtttctccaccacaatttacacagcactgagctgcaaactctaggccagcaggatgggtaa

SEQ ID NO: 17 - protein, 900 aa, Homo sapiens
  MISC_FEATURE (1)..(900): Human FAMA190A wild type
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKDIMKDECSMLKLQLKEKDELISQLQEELGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 18 - protein, 846 aa, Homo sapiens
  MISC_FEATURE (1)..(846): The amino acid sequence of the variant lacking exons 7 and 8.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQEGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 19 - protein, 820 aa, Homo sapiens
  MISC_FEATURE (1)..(820): The amino acid sequence of the variant lacking exons 7, 8 and 9.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQEGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 20 - protein, 805 aa, Homo sapiens
  MISC_FEATURE (1)..(805): The amino acid sequence of the variant lacking exons 7, 8, 9 and 10.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQEATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 21 - protein, 846 aa, Homo sapiens
  MISC_FEATURE (1)..(846): The amino acid sequence of the variant lacking exons 8 and 9.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 22 - protein, 831 aa, Homo sapiens
  MISC_FEATURE (1)..(831): The amino acid sequence of the variant lacking exons 8, 9 and 10.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 23 - protein, 859 aa, Homo sapiens
  MISC_FEATURE (1)..(859): The amino acid sequence of the variant lacking exons 9 and 10.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKDIMKDECSMLKLQLKEKDELISQLQEELATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 24 - protein, 874 aa, Homo sapiens
  MISC_FEATURE (1)..(874): The amino acid sequence of the variant lacking only exons 9.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKDIMKDECSMLKLQLKEKDELISQLQEELGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 25 - protein, 885 aa, Homo sapiens
  MISC_FEATURE (1)..(885): The amino acid sequence of the variant lacking only exons 10.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKDIMKDECSMLKLQLKEKDELISQLQEELGKVRHLQKAFASRVDKSTQTELLCYDATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 26 - protein, 874 aa, Homo sapiens
  MISC_FEATURE (1)..(874): The amino acid sequence of the variant lacking only exons 7.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQEDIMKDECSMLKLQLKEKDELISQLQEELGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 27 - protein, 872 aa, Homo sapiens
  MISC_FEATURE (1)..(872): The amino acid sequence of the variant lacking only exons 8.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 28 - DNA, 2688 bp, Mus musculus
  misc_feature (1)..(2688): Murine FAM190A wild type
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaaggatataacaagagatgaatgctcgatgctgaggctacaactgaaggacagggatgagctcatttctcagcttcaggcagagctggaaaaggtgcagcatttacagaaggcgtttgcttcaagagtagataaatccacgcagactgaacttctaggatgcgatggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 29 - DNA, 2529 bp, Mus musculus
  misc_feature (1)..(2529): Murine exon 6 joined to exon 9.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagaggaaaaggtgcagcatttacagaaggcgtttgcttcaagagtagataaatccacgcagactgaacttctaggatgcgatggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 30 - DNA, 2451 bp, Mus musculus
  misc_feature (1)..(2451): Murine exon 6 joined to exon 10.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 31 - DNA, 2406 bp, Mus musculus
  misc_feature (1)..(2406): Murine exon 6 joined to exon 11.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 32 - DNA, 2526 bp, Mus musculus
  misc_feature (1)..(2526): Murine exon 7 joined to exon 10.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaagggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 33 - DNA, 2481 bp, Mus musculus
  misc_feature (1)..(2481): Murine exon 7 joined to exon 11.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 34 - DNA, 2565 bp, Mus musculus
  misc_feature (1)..(2565): Murine exon 8 joined to exon 11.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaaggatataacaagagatgaatgctcgatgctgaggctacaactgaaggacagggatgagctcatttctcagcttcaggcagagctgactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 35 - DNA, 2610 bp, Mus musculus
  misc_feature (1)..(2610): Murine 8 joined to exon 10.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaaggatataacaagagatgaatgctcgatgctgaggctacaactgaaggacagggatgagctcatttctcagcttcaggcagagctgggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 36 - DNA, 2643 bp, Mus musculus
  misc_feature (1)..(2643): Murine exon 9 joined to exon 11.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaaggatataacaagagatgaatgctcgatgctgaggctacaactgaaggacagggatgagctcatttctcagcttcaggcagagctggaaaaggtgcagcatttacagaaggcgtttgcttcaagagtagataaatccacgcagactgaacttctaggatgcgatactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 37 - DNA, 2613 bp, Mus musculus
  misc_feature (1)..(2613): Murine exon 6 joined to exon 8.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagaggatataacaagagatgaatgctcgatgctgaggctacaactgaaggacagggatgagctcatttctcagcttcaggcagagctggaaaaggtgcagcatttacagaaggcgtttgcttcaagagtagataaatccacgcagactgaacttctaggatgcgatggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 38 - DNA, 2604 bp, Mus musculus
  misc_feature (1)..(2604): Murine exon 7 joined to exon 9.
atgggggactcaggatcaagacgatgtaccctggtctcccggttgccaatattcaggaaaagtattaacagaagacatgattctctgccttcttcaccttcctccagcaacaccgctggtgtgcatagctcctccccttccagcactaactcgagctcagggagcacgggaaaacgcaggagcatcttccgtgctccctctatcagcttccaccataagaagggcagtgaacctaagccagaacccactgagcagaaccttagtatttccaatggtgctcaacccagtcacagcaatatgcagaaactaagtttggaggaacacgtcaaaaccaggggaagacactctgtgggtttcagcagttcacgaagtaagaagataacaaggtctttgacggaagattttgaaagggaaaaggagccttcaactaataagaatgtctttataaactgtctaagttctggcagaagtgaaggggacgattctggattcacagaagaacaaagtcgccgttctattaaacagtccaccaagaagctgctccccaagtccttctcatctcactataagttttgtaagtcggttcctcagagccaatccacttcattgatacagcagcctgagttctcgctggcaattgcacaataccaagagcaggaagccgccttagggagaccttccccatcttgctctgtggatgtaacagagcgcgcaggaagttccttacagtcgcctctgctttctgctgacctgaccacagctcagaccccttcagaatttttggccttgactgaagattctttgtctgaggcagatgcctttcctaaaagtggaagcacggcatcacactgtgacaattttggccacaatgatgctacctctcagcccacttccagtcttactgctgtctcaaagacaaagatggaatttgtggggactgcgccctgtgtgatgtctccagggaggtacaggctagaaggtcggtgcagcactgaactgcattcttcaccagagacccctgctggtaaccggagggaagtgtcactacaaagcactgagctctctgttgggaatgggagtgatccagagacccatttaccggcccatcatcaaagaggagagagccccttggcacacgctggtgaaccagcgctgaggacaggctcgcccaggacactgggctcctatgaccagcacaaggcattggctgaacgctttaaaggggtccaccctgtctcggattcaagggtaatcccttcctctggggatcatgtttttaacaaaacgtcttatggatatgaagcgagtgctgccaaagttcttgccagtagcctcagtccataccgagaaggaagatatatagagcggcggctgcggtcctcctctgaagggacagcggggagcagcagaatggtcttaaagccgaaggatgggcatgtggaagccagcagcttgagaaagcacagaacggggtcctcctcttccaaaatgaacagtctggacgttttaaatcatctgggatcctgtgaactggatgaagatgacttaatgcttgatttagaatttttagaggaacagaatcttcaacccccagtttgccgggaagattcctgccactctgtaatgtcctgtacagccgttcttctcagcccagtggatccaggaaaagaagtgaatatgctagaagaaccaaaatgtcctgagccttccaaacagaacctttccctgagaataacaaaagacactgatcaagaggcccggtgttcccatgtcagctgcatgcccaacagtccctctgcagattggccccagcagggtgtggaggaaaatggaggcatagattctctgccattcagactgatgctccaggagtgcacggcagtgaagacgctgcttctgaagatgaagagagtgctgcaagagagtgatgtgagcccttccagcagtaccacctcacttcccatcagccctctgactgaagagccactgcctttcaaggaaaaggtgcagcatttacagaaggcgtttgcttcaagagtagataaatccacgcagactgaacttctaggatgcgatggtttaagcctgaaacgactggaggctgtacaaggagggagagagactacacaccgaaatcgaaccatgagtcaaagtcacagcacaagggacagaaaagcaatacatactcccaccgaggaccgttttagatattctacagctgaccagacaagtccctacaagaatatctgccaactcccaggtctgtgtcttagcaatttcttgaaggacaaggaattaggaggagttatgaaacacacaagggggaaccacgaagctgtcacatcagaaatgactcagaactcaaggacaactatgggacagagctttctgaaggcagcagctaagccagaagggctgcccatgttctcagaaaaaccaaaggacccagcagctctgtcccgacagcactccaccttcacaggcagatttggacagccacccagaggacctatctccttacacacatacagcaggaagaatgtttttctccaccacaatttacatacaacagagttccagactctgggccagcaggatgggtag

SEQ ID NO: 39 - protein, 842 aa, Mus musculus
  misc_feature (1)..(842): Murine exon 6 joined to exon 9.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQEEKVQHLQKAFASRVDKSTQTELLGCDGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 40 - protein, 816 aa, Mus musculus
  MISC_FEATURE (1)..(816): Murine exon 6 joined to exon 10.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQEGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 41 - protein, 801 aa, Mus musculus
  MISC_FEATURE (1)..(801): Murine exon 6 joined to exon 11.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 42 - protein, 841 aa, Mus musculus
  MISC_FEATURE (1)..(841): Murine exon 7 joined to exon 10.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQESDVSPSSSTTSLPISPLTEEPLPFKGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 43 - protein, 826 aa, Mus musculus
  MISC_FEATURE (1)..(826): Murine exon 7 joined to exon 11.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQESDVSPSSSTTSLPISPLTEEPLPFKTTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 44 - protein, 854 aa, Mus musculus
  MISC_FEATURE (1)..(854): Murine exon 8 joined to exon 11.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQESDVSPSSSTTSLPISPLTEEPLPFKDITRDECSMLRLQLKDRDELISQLQAELTTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 45 - protein, 869 aa, Mus musculus
  MISC_FEATURE (1)..(869): Murine 8 joined to exon 10.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQESDVSPSSSTTSLPISPLTEEPLPFKDITRDECSMLRLQLKDRDELISQLQAELGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 46 - protein, 869 aa, Mus musculus; identical to SEQ ID NO: 45
  MISC_FEATURE (1)..(869): Murine exon 9 joined to exon 11.

SEQ ID NO: 47 - protein, 870 aa, Mus musculus
  MISC_FEATURE (1)..(870): Murine exon 6 joined to exon 8.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQEDITRDECSMLRLQLKDRDELISQLQAELEKVQHLQKAFASRVDKSTQTELLGCDGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 48 - protein, 867 aa, Mus musculus
  MISC_FEATURE (1)..(867): Murine exon 7 joined to exon 9.
MGDSGSRRCTLVSRLPIFRKSINRRHDSLPSSPSSSNTAGVHSSSPSSTNSSSGSTGKRRSIFRAPSISFHHKKGSEPKPEPTEQNLSISNGAQPSHSNMQKLSLEEHVKTRGRHSVGFSSSRSKKITRSLTEDFEREKEPSTNKNVFINCLSSGRSEGDDSGFTEEQSRRSIKQSTKKLLPKSFSSHYKFCKSVPQSQSTSLIQQPEFSLAIAQYQEQEAALGRPSPSCSVDVTERAGSSLQSPLLSADLTTAQTPSEFLALTEDSLSEADAFPKSGSTASHCDNFGHNDATSQPTSSLTAVSKTKMEFVGTAPCVMSPGRYRLEGRCSTELHSSPETPAGNRREVSLQSTELSVGNGSDPETHLPAHHQRGESPLAHAGEPALRTGSPRTLGSYDQHKALAERFKGVHPVSDSRVIPSSGDHVFNKTSYGYEASAAKVLASSLSPYREGRYIERRLRSSSEGTAGSSRMVLKPKDGHVEASSLRKHRTGSSSSKMNSLDVLNHLGSCELDEDDLMLDLEFLEEQNLQPPVCREDSCHSVMSCTAVLLSPVDPGKEVNMLEEPKCPEPSKQNLSLRITKDTDQEARCSHVSCMPNSPSADWPQQGVEENGGIDSLPFRLMLQECTAVKTLLLKMKRVLQESDVSPSSSTTSLPISPLTEEPLPFKEKVQHLQKAFASRVDKSTQTELLGCDGLSLKRLEAVQGGRETTHRNRTMSQSHSTRDRKAIHTPTEDRFRYSTADQTSPYKNICQLPGLCLSNFLKDKELGGVMKHTRGNHEAVTSEMTQNSRTTMGQSFLKAAAKPEGLPMFSEKPKDPAALSRQHSTFTGRFGQPPRGPISLHTYSRKNVFLHHNLHTTEFQTLGQQDG

SEQ ID NO: 49 - protein, 733 aa, Homo sapiens
  MISC_FEATURE (1)..(733): The amino acid sequence of the variant lacking exons 4-7.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDIMKDECSMLKLQLKEKDELISQLQEELGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 50 - protein, 759 aa, Homo sapiens
  MISC_FEATURE (1)..(759): The amino acid sequence of the variant lacking exons 4-6.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLSADMSPASSTTSLPVSPLTEEPVPFKDIMKDECSMLKLQLKEKDELISQLQEELGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 51 - protein, 705 aa, Homo sapiens
  MISC_FEATURE (1)..(705): The amino acid sequence of the variant lacking exons 4-8.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 52 - protein, 679 aa, Homo sapiens
  MISC_FEATURE (1)..(679): The amino acid sequence of the variant lacking exons 4-9.
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 53 - protein, 679 aa, Homo sapiens; identical to SEQ ID NO: 52
  MISC_FEATURE (1)..(679): The amino acid sequence of the variant lacking exons 4-10.

SEQ ID NO: 54 - protein, 20 aa, Homo sapiens
  misc_feature (20)..(20): Xaa can be any naturally occurring amino acid
SSSSKMNSLESFPEINKGRX

SEQ ID NO: 55 - protein, 13 aa, Homo sapiens
  misc_feature (13)..(13): Xaa can be any naturally occurring amino acid
PEFPEPSKQVQTX

SEQ ID NO: 56 - protein, 18 aa, Homo sapiens
LKMKRVLQEDIMKDECSM

SEQ ID NO: 57 - protein, 18 aa, Homo sapiens
LKMKRVLQEGKVRHLQKA

SEQ ID NO: 58 - protein, 18 aa, Homo sapiens
LKMKRVLQEGLNLKRLET

SEQ ID NO: 59 - protein, 18 aa, Homo sapiens
LKMKRVLQEATYRNRIVS

SEQ ID NO: 60 - protein, 46 aa, Homo sapiens
LKMKRVLQEDIMKDECSMLKLQLKEKDELISQLQEELGLNLKRLET

SEQ ID NO: 61 - protein, 18 aa, Homo sapiens
LTEEPVPFKGKVRHLQKA

SEQ ID NO: 62 - protein, 18 aa, Homo sapiens
LTEEPVPFKGLNLKRLET

SEQ ID NO: 63 - protein, 18 aa, Homo sapiens
LTEEPVPFKATYRNRIVS

SEQ ID NO: 64 - protein, 18 aa, Homo sapiens
LISQLQEELGLNLKRLET

SEQ ID NO: 65 - protein, 18 aa, Homo sapiens
LISQLQEELATYRNRIVS

SEQ ID NO: 66 - protein, 18 aa, Homo sapiens
TQTELLCYDATYRNRIVS

SEQ ID NO: 67 - DNA, 20 bp, Artificial Sequence
  Primer
gatgaatgctcgatgctcaa

SEQ ID NO: 68 - DNA, 20 bp, Artificial Sequence
  Primer
tcctgctggcctagagtttg

SEQ ID NO: 69 - DNA, 20 bp, Artificial Sequence
  Primer
gcagacatgagtccagcaag

SEQ ID NO: 70 - DNA, 20 bp, Artificial Sequence
  Primer
cgtctgaagtgagggtttca

SEQ ID NO: 71 - DNA, 20 bp, Artificial Sequence
  Primer
gagtccagcaagcagtacca

SEQ ID NO: 72 - DNA, 20 bp, Artificial Sequence
  Primer
agaacgtggagagcccttct

SEQ ID NO: 73 - DNA, 20 bp, Artificial Sequence
  Primer
ggcctctacaaggtgtggaa

SEQ ID NO: 74 - DNA, 20 bp, Artificial Sequence
  Primer
ccgtagcaacttggtccttt

SEQ ID NO: 75 - DNA, 20 bp, Artificial Sequence
  Primer
gtggcccgtaagttctgtgt

SEQ ID NO: 76 - DNA, 20 bp, Artificial Sequence
  Primer
ttttgtaggggcttgtctgg

SEQ ID NO: 77 - DNA, 20 bp, Artificial Sequence
  Primer
gaatgcccaacagtccatct

SEQ ID NO: 78 - DNA, 20 bp, Artificial Sequence
  Primer
ggcctctacaaggtgtggaa

SEQ ID NO: 79 - protein, 6 aa, Homo sapiens
LQEGKV

SEQ ID NO: 80 - protein, 6 aa, Homo sapiens
LQEGLN

SEQ ID NO: 81 - protein, 6 aa, Homo sapiens
LQEATY

SEQ ID NO: 82 - protein, 6 aa, Homo sapiens
PFKGLN

SEQ ID NO: 83 - protein, 6 aa, Homo sapiens
PFKATY

SEQ ID NO: 84 - protein, 6 aa, Homo sapiens
EELATY

SEQ ID NO: 85 - protein, 6 aa, Homo sapiens
EELGLN

SEQ ID NO: 86 - protein, 6 aa, Homo sapiens
CYDATY

SEQ ID NO: 87 - protein, 6 aa, Homo sapiens
NSLDIM

SEQ ID NO: 88 - protein, 6 aa, Homo sapiens
NSLSAD

SEQ ID NO: 89 - protein, 6 aa, Homo sapiens
NSLGKV

SEQ ID NO: 90 - protein, 6 aa, Homo sapiens
NSLGLN

SEQ ID NO: 91 - protein, 6 aa, Homo sapiens
NSLATY

SEQ ID NO: 92 - protein, 6 aa, Homo sapiens
LQEDIM

SEQ ID NO: 93 - protein, 6 aa, Homo sapiens
PFKGKV

SEQ ID NO: 94 - protein, 6 aa, Homo sapiens
NSLESF

SEQ ID NO: 95 - protein, 5 aa, Homo sapiens
SKQVQ

SEQ ID NO: 96 - DNA, 20 bp, Artificial Sequence
  Primer
atccgcctctcctctctctc

SEQ ID NO: 97 - DNA, 20 bp, Artificial Sequence
  Primer
cccttcacttttgccagaac

SEQ ID NO: 98 - DNA, 20 bp, Artificial Sequence
  Primer
tgaaggggatgattctggtt

SEQ ID NO: 99 - DNA, 20 bp, Artificial Sequence
  Primer
ccaagtttccttggggagtt

SEQ ID NO: 100 - DNA, 20 bp, Artificial Sequence
  Primer
ccccaaggaaacttggattt

SEQ ID NO: 101 - DNA, 20 bp, Artificial Sequence
  Primer
gaaaggcactggctcttcag

SEQ ID NO: 102 - DNA, 20 bp, Artificial Sequence
  Primer
gatgaatgctcgatgctcaa

SEQ ID NO: 103 - DNA, 20 bp, Artificial Sequence
  Primer
tcctgctggcctagagtttg

SEQ ID NO: 104 - DNA, 23 bp, Artificial Sequence
  Primer
agcaatctgttcactgttaatgg

SEQ ID NO: 105 - DNA, 20 bp, Artificial Sequence
  Primer
tttgccatcaaatgggtttt

SEQ ID NO: 106 - DNA, 20 bp, Artificial Sequence
  Primer
tttgacaaataaggcccaaa

SEQ ID NO: 107 - DNA, 20 bp, Artificial Sequence
  Primer
taaatattgcccagcccaaa

SEQ ID NO: 108 - DNA, 20 bp, Artificial Sequence
  Primer
caggcctggagaaggtgtaa

SEQ ID NO: 109 - DNA, 20 bp, Artificial Sequence
  Primer
ggggcaactatgcctacaaa

SEQ ID NO: 110 - DNA, 21 bp, Artificial Sequence
  Primer
tttgcgtgtttgtaattctgc

SEQ ID NO: 111 - DNA, 20 bp, Artificial Sequence
  Primer
cactgaaagaatgcccacat

SEQ ID NO: 112 - DNA, 20 bp, Artificial Sequence
  Primer
cctttacaggcaggtttgga

SEQ ID NO: 113 - DNA, 20 bp, Artificial Sequence
  Primer
caagcaagccacattcttca

SEQ ID NO: 114 - DNA, 20 bp, Artificial Sequence
  Primer
tgaagaatgtggcttgcttg

SEQ ID NO: 115 - DNA, 22 bp, Artificial Sequence
  Primer
ttgagtgctctttttccctttc

SEQ ID NO: 116 - DNA, 20 bp, Artificial Sequence
  Primer
aggggaagacattctgttgg

SEQ ID NO: 117 - DNA, 20 bp, Artificial Sequence
  Primer
gagtttgtccccagcattgt

SEQ ID NO: 118 - DNA, 20 bp, Artificial Sequence
  Primer
gaaggcactgcagggagtag

SEQ ID NO: 119 - DNA, 20 bp, Artificial Sequence
  Primer
gcagacatgagtccagcaag

SEQ ID NO: 120 - DNA, 20 bp, Artificial Sequence
  Primer
gtcagccgtaagctgaaacc

SEQ ID NO: 121 - DNA, 20 bp, Artificial Sequence
  Primer
gggagaccagggtagatcgt

SEQ ID NO: 122 - DNA, 20 bp, Artificial Sequence
  Primer
acaatgctggggacaaactc

SEQ ID NO: 123 - DNA, 20 bp, Artificial Sequence
  Primer
tactccctgcagtgccttct

SEQ ID NO: 124 - DNA, 20 bp, Artificial Sequence
  Primer
cccaacttcaggaagagctg

SEQ ID NO: 125 - DNA, 20 bp, Artificial Sequence
  Primer
ttttgtaggggcttgtctgg

SEQ ID NO: 126 - DNA, 20 bp, Artificial Sequence
  Primer
aatctgtcagcgtgcacaag

SEQ ID NO: 127 - DNA, 20 bp, Artificial Sequence
  Primer
gcacatgtgtgttcctttgg

SEQ ID NO: 128 - DNA, 21 bp, Artificial Sequence
  Primer
catctaaagttcccaccatac

SEQ ID NO: 129 - DNA, 21 bp, Artificial Sequence
  Primer
acagacaccaacatttaggac

SEQ ID NO: 130 - DNA, 20 bp, Artificial Sequence
  Primer
gaatgcccaacagtccatct

SEQ ID NO: 131 - DNA, 20 bp, Artificial Sequence
  Primer
cgtctgaagtgagggtttca

SEQ ID NO: 132 - DNA, 20 bp, Artificial Sequence
  Primer
gagtccagcaagcagtacca

SEQ ID NO: 133 - DNA, 20 bp, Artificial Sequence
  Primer
agaacgtggagagcccttct

SEQ ID NO: 134 - DNA, 20 bp, Artificial Sequence
  Primer
ggcctctacaaggtgtggaa

SEQ ID NO: 135 - DNA, 20 bp, Artificial Sequence
  Primer
ccgtagcaacttggtccttt

SEQ ID NO: 136 - DNA, 20 bp, Artificial Sequence
  Primer
gtggcccgtaagttctgtgt

SEQ ID NO: 137 - DNA, 20 bp, Artificial Sequence
  Primer
gggcatttacccagacagaa

SEQ ID NO: 138 - DNA, 20 bp, Artificial Sequence
  Primer
tttgcagcctgagaaaggtt

SEQ ID NO: 139 - DNA, 20 bp, Artificial Sequence
  Primer
acaatgctggggacaaactc

SEQ ID NO: 140 - DNA, 20 bp, Artificial Sequence
  Primer
aaaatcaagcaggggaaaca

SEQ ID NO: 141 - DNA, 20 bp, Artificial Sequence
  Primer
atcctgccaaaggtatgctg

SEQ ID NO: 142 - DNA, 21 bp, Artificial Sequence
  Primer
tttcttctgaaggtgctccaa

SEQ ID NO: 143 - DNA, 20 bp, Artificial Sequence
  Primer
gtgagcaacgaagcaacaaa

SEQ ID NO: 144 - DNA, 20 bp, Artificial Sequence
  Primer
tactccctgcagtgccttct

SEQ ID NO: 145 - DNA, 20 bp, Artificial Sequence
  Primer
atcctcgtcagaaggcactg

SEQ ID NO: 146 - DNA, 21 bp, Artificial Sequence
  Primer
tggaaacaaattttcccttca

SEQ ID NO: 147 - DNA, 21 bp, Artificial Sequence
  Primer
aagcaaagagcaggttcttca

SEQ ID NO: 148 - DNA, 20 bp, Artificial Sequence
  Primer
tccatggaagcaaggaagac

SEQ ID NO: 149 - DNA, 20 bp, Artificial Sequence
  Primer
catttttgcttcacggcttc

SEQ ID NO: 150 - DNA, 22 bp, Artificial Sequence
  Primer
gaaagctactgaccagggtagg

SEQ ID NO: 151 - DNA, 20 bp, Artificial Sequence
  Primer
ttgggatcttgtgaactgga

SEQ ID NO: 152 - DNA, 20 bp, Artificial Sequence
  Primer
cgcacccaaattatgtctcc

SEQ ID NO: 153 - DNA, 20 bp, Artificial Sequence
  Primer
aatgacttgagggagcctga

SEQ ID NO: 154 - DNA, 20 bp, Artificial Sequence
  Primer
gcagtaggtcccaacgttct

SEQ ID NO: 155 - DNA, 20 bp, Artificial Sequence
  Primer
tgttttcctgcccattcttc

SEQ ID NO: 156 - DNA, 21 bp, Artificial Sequence
  Primer
aaaggaatttgctcactgcaa

SEQ ID NO: 157 - DNA, 20 bp, Artificial Sequence
  Primer
gaaaaattgcccaaggtcat

SEQ ID NO: 158 - DNA, 20 bp, Artificial Sequence
  Primer
aactactgcggcacatgaga

SEQ ID NO: 159 - DNA, 21 bp, Artificial Sequence
  Primer
aaggggaaaaaggagatgtga

SEQ ID NO: 160 - DNA, 20 bp, Artificial Sequence
  Primer
ctcactgtttggaaggctca

SEQ ID NO: 161 - DNA, 20 bp, Artificial Sequence
  Primer
aggaaggagaatcccttgga

SEQ ID NO: 162 - DNA, 20 bp, Artificial Sequence
  Primer
tttgcaacatgaccacacct

SEQ ID NO: 163 - DNA, 20 bp, Artificial Sequence
  Primer
ggtgtggtcatgttgcaaag

SEQ ID NO: 164 - DNA, 20 bp, Artificial Sequence
  Primer
actgtttggaaggctcagga

SEQ ID NO: 165 - DNA, 20 bp, Artificial Sequence
  Primer
cgttgatcaagaagccaggt

SEQ ID NO: 166 - DNA, 20 bp, Artificial Sequence
  Primer
tgctctaagcatgctgcact

SEQ ID NO: 167 - DNA, 20 bp, Artificial Sequence
  Primer
gagcagggtctaaccacgtc

SEQ ID NO: 168 - DNA, 20 bp, Artificial Sequence
  Primer
gaatgcccaacagtccatct

SEQ ID NO: 169 - DNA, 20 bp, Artificial Sequence
  Primer
gtgcagacatgagtccagca

SEQ ID NO: 170 - DNA, 23 bp, Artificial Sequence
  Primer
atttgaaatcagacccttgaaga

SEQ ID NO: 171 - DNA, 27 bp, Artificial Sequence
  Primer
tgcacttaaaatatacatggagagtga

SEQ ID NO: 172 - DNA, 20 bp, Artificial Sequence
  Primer
gaaaggcactggctcttcag

SEQ ID NO: 173 - DNA, 20 bp, Artificial Sequence
  Primer
ctcccactgtgtgaagctga

SEQ ID NO: 174 - DNA, 20 bp, Artificial Sequence
  Primer
ggattgaaccactggaagga

SEQ ID NO: 175 - DNA, 23 bp, Artificial Sequence
  Primer
tcaagagcaaagtaaaagggcta

SEQ ID NO: 176 - DNA, 23 bp, Artificial Sequence
  Primer
aaccgaaatgtttaatcctggtt

SEQ ID NO: 177 - DNA, 20 bp, Artificial Sequence
  Primer
gatgaatgctcgatgctcaa

SEQ ID NO: 178 - DNA, 20 bp, Artificial Sequence
  Primer
acctgaaccacacacactgc

SEQ ID NO: 179 - DNA, 20 bp, Artificial Sequence
  Primer
cccaacttcaggaagagctg

SEQ ID NO: 180 - DNA, 20 bp, Artificial Sequence
  Primer
aatgtggcctaagccttcaa

SEQ ID NO: 181 - DNA, 20 bp, Artificial Sequence
  Primer
aaggtgggacagttgtgacc

SEQ ID NO: 182 - DNA, 20 bp, Artificial Sequence
  Primer
ccaggaggtatttggggatt

SEQ ID NO: 183 - DNA, 20 bp, Artificial Sequence
  Primer
cattttgcagggaaaagtcc

SEQ ID NO: 184 - DNA, 20 bp, Artificial Sequence
  Primer
gtgcaccgctaaatcctgtt

SEQ ID NO: 185 - DNA, 20 bp, Artificial Sequence
  Primer
tactcagggccatcgcttag

SEQ ID NO: 186 - DNA, 20 bp, Artificial Sequence
  Primer
taattggagcaacgggagtc

SEQ ID NO: 187 - DNA, 20 bp, Artificial Sequence
  Primer
gtactgcaagggcttctgct

SEQ ID NO: 188 - DNA, 20 bp, Artificial Sequence
  Primer
gcgctgagatctgctggtag

SEQ ID NO: 189 - DNA, 20 bp, Artificial Sequence
  Primer
tgctttggcagaatgaacag

SEQ ID NO: 190 - DNA, 20 bp, Artificial Sequence
  Primer
caggaaatgcattgggacta

SEQ ID NO: 191 - DNA, 20 bp, Artificial Sequence
  Primer
ttttcccacccctcttcttt

SEQ ID NO: 192 - DNA, 27 bp, Artificial Sequence
  Primer
ggctcttgcttaggctcactccccttc

SEQ ID NO: 193 - DNA, 26 bp, Artificial Sequence
  Primer
tattggcaaccgggagaccagggtag

SEQ ID NO: 194 - DNA, 20 bp, Artificial Sequence
  Primer
cccttcacttttgccagaac

SEQ ID NO: 195 - DNA, 20 bp, Artificial Sequence
  Primer
ccagtggatccaggaaaaga

SEQ ID NO: 196 - DNA, 20 bp, Artificial Sequence
  Primer
tggttcgatttcggtgtgta

SEQ ID NO: 197 - DNA, 20 bp, Artificial Sequence
  Primer
cttgccagtagcctcagtcc

SEQ ID NO: 198 - DNA, 20 bp, Artificial Sequence
  Primer
cagaagcagcgtcttcactg

SEQ ID NO: 199 - DNA, 20 bp, Artificial Sequence
  Primer
cgctcagaatgactgactgc

SEQ ID NO: 200 - DNA, 20 bp, Artificial Sequence
  Primer
cctggttttgacgtgttcct

SEQ ID NO: 201 - DNA, 20 bp, Artificial Sequence
  Primer
gcaggaggagtgctgaagac

SEQ ID NO: 202 - DNA, 20 bp, Artificial Sequence
  Primer
tgtcttcccctggttttgac

SEQ ID NO: 203 - DNA, 20 bp, Artificial Sequence
  Primer
accacctcacttcccatcag

SEQ ID NO: 204 - DNA, 20 bp, Artificial Sequence
  Primer
tgggtcctttggtttttctg

SEQ ID NO: 205 - DNA, 20 bp, Artificial Sequence
  Primer
gatggggaaggtctccctaa

SEQ ID NO: 206 - DNA, 20 bp, Artificial Sequence
  Primer
tagggagaccttccccatct

SEQ ID NO: 207 - DNA, 20 bp, Artificial Sequence
  Primer
ccagttcacaggatcccaga

SEQ ID NO: 208 - DNA, 20 bp, Artificial Sequence
  Primer
aacgctttaaaggggtccac

SEQ ID NO: 209 - DNA, 20 bp, Artificial Sequence
  Primer
tcttgaagcaaacgccttct

SEQ ID NO: 210 - DNA, 20 bp, Artificial Sequence
  Primer
tgccattcagactgatgctc

SEQ ID NO: 211 - DNA, 20 bp, Artificial Sequence
  Primer
gggcatgaccactcttaggt

SEQ ID NO: 212 - DNA, 20 bp, Artificial Sequence
  Primer
agagtgatgtgagcccttcc

SEQ ID NO: 213 - DNA, 24 bp, Artificial Sequence
  Primer
tcagtcaacatactcaagctgtca

SEQ ID NO: 214 - DNA, 20 bp, Artificial Sequence
  Primer
agtgaagacgctgcttctga

SEQ ID NO: 215 - DNA, 21 bp, Artificial Sequence
  Primer
aaaagcccataagtctttgct

SEQ ID NO: 216 - DNA, 20 bp, Artificial Sequence
  Primer
ctgtattcccctccatcgtg

SEQ ID NO: 217 - DNA, 20 bp, Artificial Sequence
  Primer
aaggaaggctggaaaagagc

SEQ ID NO: 218 - DNA, 12 bp, Homo sapiens
caagagggaaaa

SEQ ID NO: 219 - protein, 4 aa, Homo sapiens
QEGK

SEQ ID NO: 220 - DNA, 12 bp, Homo sapiens
caagagggttta

SEQ ID NO: 221 - protein, 4 aa, Homo sapiens
QEGL

SEQ ID NO: 222 - DNA, 12 bp, Homo sapiens
caagaggctaca

SEQ ID NO: 223 - protein, 4 aa, Homo sapiens
QEAT

SEQ ID NO: 224 - DNA, 12 bp, Homo sapiens
ttcaagggttta

SEQ ID NO: 225 - protein, 4 aa, Homo sapiens
FKGL

SEQ ID NO: 226 - DNA, 12 bp, Homo sapiens
ttcaaggctaca

SEQ ID NO: 227 - protein, 4 aa, Homo sapiens
FKAT

SEQ ID NO: 228 - DNA, 12 bp, Homo sapiens
gagctggctaca

SEQ ID NO: 229 - protein, 4 aa, Homo sapiens
ELAT

SEQ ID NO: 230 - DNA, 12 bp, Homo sapiens
gagctgggttta

SEQ ID NO: 231 - protein, 4 aa, Homo sapiens
ELGL

SEQ ID NO: 232 - DNA, 12 bp, Homo sapiens
tatgatgctaca

SEQ ID NO: 233 - protein, 4 aa, Homo sapiens
YDAT

SEQ ID NO: 234 - DNA, 12 bp, Homo sapiens
agtttggatata

SEQ ID NO: 235 - protein, 4 aa, Homo sapiens
SLDI

SEQ ID NO: 236 - DNA, 12 bp, Homo sapiens
agtttgagtgca

SEQ ID NO: 237 - protein, 4 aa, Homo sapiens
SLSA

SEQ ID NO: 238 - DNA, 12 bp, Homo sapiens
agtttgggaaaa

SEQ ID NO: 239 - protein, 4 aa, Homo sapiens
SLGK

SEQ ID NO: 240 - DNA, 12 bp, Homo sapiens
agtttgggttta

SEQ ID NO: 241 - protein, 4 aa, Homo sapiens
SLGL

SEQ ID NO: 242 - DNA, 12 bp, Homo sapiens
agtttggctaca

SEQ ID NO: 243 - protein, 4 aa, Homo sapiens
SLAT

SEQ ID NO: 244 - DNA, 12 bp, Homo sapiens
caagaggatata

SEQ ID NO: 245 - protein, 4 aa, Homo sapiens
QEDI

SEQ ID NO: 246 - DNA, 11 bp, Homo sapiens
ttcaagggaaa

SEQ ID NO: 247 - protein, 4 aa, Homo sapiens
FKGK

SEQ ID NO: 248 - DNA, 14 bp, Homo sapiens
tactatgctatgat

SEQ ID NO: 249 - DNA, 14 bp, Homo sapiens
ggtttaaacttgaa

SEQ ID NO: 250 - DNA, 14 bp, Homo sapiens
ttcaggaagagctg

SEQ ID NO: 251 - DNA, 14 bp, Homo sapiens
gctacatatgcaaa

SEQ ID NO: 252 - DNA, 14 bp, Homo sapiens
gagttcttcaagag

SEQ ID NO: 253 - DNA, 14 bp, Homo sapiens
gctacatatgcaaa

SEQ ID NO: 254 - DNA, 14 bp, Homo sapiens
ttcaggaagagctg

SEQ ID NO: 255 - DNA, 14 bp, Homo sapiens
ggtttaaacttgaa

SEQ ID NO: 256 - DNA, 14 bp, Homo sapiens
cagtgcctttcaag

SEQ ID NO: 257 - DNA, 14 bp, Homo sapiens
gctacatatgcaaa

SEQ ID NO: 258 - DNA, 14 bp, Homo sapiens
tactatgctatgat

SEQ ID NO: 259 - DNA, 14 bp, Homo sapiens
gctacatatgcaaa